(19) United States
(12) Patent Application Publication
(10) Pub. No.: US 2006/0130161 A1
(43) Pub. Date: Jun. 15, 2006

(54) Animal model systems for viral pathogenesis of neurodegeneration, autoimmune demyelination, and diabetes

(75) Inventor: Claude Genain, Mill Valley, CA (US)

Correspondence Address: SPECKMAN LAW GROUP PLLC, 1201 THIRD AVENUE, SUITE 330, SEATTLE, WA, 98101, US

(73) Assignee: CARANTECH, INC., Mill Valley, CA (US)

(21) Appl. No.: 11/248,499

(22) Filed: Oct. 12, 2005

Related U.S. Application Data
(60) Provisional application No. 60/618,277, filed on Oct. 12, 2004.
(60) Provisional application No. 60/720,676, filed on Sep. 26, 2005.

(51) Int. Cl. A01K 67/027

(52) U.S. Cl. 800/9; 800/14

(57) ABSTRACT

Provided are non-human animal model systems for viral pathogenesis of neurodegeneration, autoimmune demyelination, and autoimmune diseases such as diseases of the central nervous system, including multiple sclerosis (MS), and diabetes. Such non-human animal model systems may be suitably employed for the study of diseases such as MS and diabetes and for the identification and characterization of candidate therapeutic compounds and compositions for the treatment of such diseases. Also provided herein are markers and methods for the detection, in patients susceptible to autoimmune disease, of autoimmune diseases of the central nervous system such as progressive multifocal leukoencephalopathy (PML) following treatment with one or more therapeutic agent as exemplified herein by the therapeutic agent natalizumab. Exemplary animal model systems comprise marmosets infected with a herpesvirus such as HHV6-A and HHV6-B, transgenic mouse and zebrafish animal model systems wherein the transgene encodes CD46, and methods for monitoring the risks of patients having MS, diabetes and other auto-immune disorders treated with anti-adhesion molecules such as natalizumab.

### CROSS-REFERENCE TO RELATED APPLICATIONS

This application claims priority under 35 U.S.C. § 119(e) to U.S. Provisional Patent Application No. 60/618,277, filed Oct. 12, 2004, and to co-pending U.S. Provisional Patent Application No. 60/720,676, filed Sep. 26, 2005, each of which is incorporated herein by reference in its entirety.

### GOVERNMENT SUPPORT

Certain aspects of the presently disclosed inventions were developed with support from a National Multiple Sclerosis Society Pilot Grant No. PP0916. The Government may have certain rights to some aspects of those inventions.

### BACKGROUND OF THE INVENTION

1. Technical Field of the Invention

The present invention relates generally to viral pathogenesis and autoimmune diseases such as diseases of the central nervous system, including multiple sclerosis (MS), and diabetes. More specifically, provided herein are non-human animal model systems for viral pathogenesis of neurodegeneration and autoimmune demyelination. Such animal model systems may be suitably employed for the study of MS and for the identification and characterization of candidate therapeutic compounds and compositions for the treatment of MS. Also provided herein are markers and methods for the detection, in patients susceptible to autoimmune disease, of autoimmune diseases of the central nervous system such as progressive multifocal leukoencephalopathy (PML) following treatment with one or more therapeutic agent as exemplified herein by the therapeutic agent natalizumab.

2. Description of the Related Art

Multiple Sclerosis (MS) designates a group of heterogeneous, immune-mediated chronic demyelinating disorders of the central nervous system (CNS) affecting 350,000 Americans and over 1 million individuals worldwide. MS affects women twice as often as men, and thus also represents a significant women's health issue. Pathologically, MS is characterized by plaques of perivascular infiltration comprised of mononuclear cells and macrophages, accompanied by concentric destruction of the myelin sheaths (demyelination), death of oligodendrocytes, proliferation of astrocytes, and axonal damage. Lassmann, Multiple Sclerosis 4:93-98 (1998); Raine, Multiple Sclerosis and Chronic Relapsing EAE: Comparative Ultrastructural Neuropathology, in Multiple Sclerosis: Pathology, Diagnosis and Management 413-460 (Hallpike et al. eds., 1983); and Trapp et al., The New England Journal of Medicine 338:278-285 (1998).

The etiology of MS is unknown; however, strong circumstantial evidence suggests that MS is an autoimmune disorder arising in a genetically susceptible host under the pressure of environmental triggers. Hohlfeld, Brain 120:865-916 (1997) and Oksenberg et al., Pathogenesis of Multiple Sclerosis: Relationship to Therapeutic Strategies, in Multiple Sclerosis: Advances in Clinical Trial Design, Treatment and Future Perspectives 14-46 (Goodkin et al. eds., 1996). To a large extent, our current knowledge of the factors that may participate in the pathogenesis of MS lesions is based on observations of experimental allergic encephalomyelitis (EAE), an autoimmune disorder that is produced in laboratory animals by sensitization with antigens of CNS myelin. Martin et al., Ann. Rev. Immunol. 10:153-187 (1992); Miller et al., Immunol. Today 15:356-361 (1994); and Wekerle et al., Ann Neurol 36:S47-S53 (1994).

In contrast to the often-stereotyped illnesses encountered in the many models of EAE, the clinical phenotype of human MS can be benign or rapidly disabling, with variable courses including relapsing, remitting, or progressive forms. This heterogeneity of clinical presentation most likely reflects complex influences of environment and/or inherited genetic factors, and may correlate with distinct neuropathological subtypes as suggested by recent analyses of biopsy and autopsy material that showed specific patterns of lesions with various proportions of inflammation, demyelination, and oligodendrocyte and axonal pathology. Lassmann, Multiple Sclerosis 4:93-98 (1998); Lucchinetti et al., Ann. Neurol. 47:707-717 (2000); and Storch et al., Ann Neurol 43:465-471 (1998). Effector mechanisms of tissue damage in CNS autoimmunity include direct toxicity of infiltrating T cells, secretion of pro-inflammatory cytokines, antibody-mediated toxicity, and complement and macrophage activation (reviewed in Brosnan et al., Brain Pathol 6:243-257 (1996)).

A viral etiology has been long suspected for MS based on epidemiologic studies (Kurtzke, Clin. Microbiol. Rev. 6:382-427 (1993); Kurtzke et al., Neurology 36:307-328 (1986)) and circumstantial evidence of CNS demyelinating diseases that occur in the context of infection with neurotropic viruses (Gilden et al., Multiple Sclerosis 2:179-183 (1996); Stohlman et al., Brain Pathol 11:92-106 (2001); Raine, in Textbook of Neuropathology 627-714 (Davis et al., eds. 1997a)). A popular hypothesis is that infections may trigger molecular mimicry, a phenomenon by which T cells of the immune system recognize a viral peptide that is the mimic of a peptide of myelin (direct mimicry). CNS invasion by T cells following viral infection, whether due to mimicry or to clear the acute infestation, may also damage the myelin and/or neurons, through either direct cytotoxicity to cells harboring the virus or production of pro-inflammatory products that create a toxic environment within the CNS and activation of macrophages or microglia (bystander damage). This in turn may trigger secondary immune attacks against exposed CNS antigens (Stohlman et al., Brain Pathol 11:92-106 (2001)).

The association between certain viral infections or vaccinations (for example measles, varicella zoster, vaccinia, Epstein Barr virus (EBV), HTLV-I) and cases of acute disseminated encephalomyelitis, encephalitis or myelitis is well recognized. It is also widely perceived that viral infections may trigger MS attacks. Higher antibody titers against neurotropic viruses are reported for MS serum or cerebrospinal fluid (CSF) compared to controls (Johnson et al., N Engl J Med 310:137-141 (1984); and Johnson, Ann Neurol 36:S54-S60 (1994)). The presence of an antigen-driven, CNS restricted immune response in MS and in infections of the CNS is supported by findings of specific oligoclonal bands in patients' CSF (Tourtelotte et al., Neurology 30:240-244 (1980)), and the more recent demonstration of clonal expansion of specific B cell immunoglobulin gene rearrangements (Baranzini et al., J. Immunol. 163:5133-44 (1999); Owens et al., An. Neurol. 43:236-243 (1998); Colombo et al., J. Immunol. 164:2782-2789 (2000); and Qin et al., J. Clin. Invest. 102:1045-1050 (1998)). In contrast to oligoclonal bands that, in CNS infections, are directed against viral antigens (Gilden et al., Multiple Sclerosis 2:179-183 (1996)), the specificity of oligoclonal bands in MS has not been established. It has, however, recently been suggested that they may react to some component of Epstein-Barr virus (Cepok et al., J Clin Invest 115:1352-60 (2005)). The number of viruses that have been incriminated in MS pathogenesis is constantly growing, and in fact interferon (IFN)-β was first tried as a treatment for MS owing to its anti-viral activity.

One difficulty in establishing direct relationships between viral exposures and MS is that appropriate in vivo experimental systems for validation of such associations are lacking. Examples of virus capable of inducing acute or chronic demyelinating disease include canine distemper virus, the JHM strain of mouse hepatitis virus, murine Semliki Forest virus, sheep Visna, caprine arthritis-encephalitis virus, SV40 in macaque monkeys, Theiler's murine encephalomyelitis virus (TMEV) (Johnson, Ann Neurol 36:S54-S60 (1994)), and lymphocytic choriomeningitis virus (LCMV) (Evans et al., Journal of Experimental Medicine 184:2371-84 (1996)). Viral proteins can also be expressed in the CNS of transgenic mice, which renders the animals susceptible to infection (Evans et al., Journal of Experimental Medicine 184:2371-84 (1996)). Disease pathogenesis varies between these models, and may include a component linked to viral persistence (monophasic disease), or secondary CNS inflammation and destruction not associated with virus infestation. Infection of mice with TMEV produces a gastroenteritis, which is rapidly cleared. Only inbred susceptible strains subsequently develop an unrelenting and severe progressive demyelinating disease with what is believed to be bystander damage to myelin. Stohlman et al., Brain Pathol 11:92-106 (2001) and Dal Canto et al., Microscopy Research and Technique 32:215-29 (1995). Infection of mice with LCMV produces a cytotoxic, CD8+ mediated response that directly destroys CNS cell targets. It is important to understand that although these models have provided the first (and only existing) insights into the relationships between autoimmune CNS demyelination and viral infections, they are still insufficient for proving direct association of MS with any of the viruses that ubiquitously infect humans without adverse consequences. CNS complications of TMEV infections are under the restrictive control of genetic influence and it is difficult to extrapolate their mechanisms to outbred human populations. Many of these disease models require intracranial injection of viruses in neonatal animals, an artificial situation that similar to EAE does not mimic natural exposure of humans to pathogens.

Immunization of Callithrix jacchus (C. jacchus) marmosets with whole human white matter, and myelin/oligodendrocyte glycoprotein (MOG) in adjuvant produce chronic, relapsing-remitting disorders of mild to moderate clinical severity which are reminiscent of typical forms of human MS. The neuropathology of acute C. jacchus EAE consists of large concentric areas of primary demyelination, macrophage infiltration, astrogliosis, and death of oligodendrocytes. Massacesi et al., Ann. Neurol. 37:519-530 (1995); Genain et al., Immunol. Reviews 183:159-172 (2001); and Brok et al., Immunol Rev 183:173-185 (2001). Ultrastructural features of myelin breakdown are similar in marmoset EAE and human MS, suggesting common mechanisms of myelin destruction. Genain et al., Immunol. Reviews 183:159-172 (2001) and Raine et al., Ann Neurol 46:144-160 (1999). Remyelination occurs in chronic EAE.

C. jacchus marmosets are small animals (350-400 gm), yet serial paraclinical and laboratory studies, such as peripheral blood reactivity to myelin antigens, CSF sampling, and in vivo magnetic resonance imaging (MRI) can be obtained. Genain et al., Proc. Natl. Acad. Sci. USA 92:3601-3605 (1995); Genain et al., Methods: a Companion to Methods in Enzymology 10:420-434 (1996); Jordan et al., AJNR Am. J. Neuroradiol. 20:965-976 (1999); and Hart et al., Am. J. Pathol. 153:649-663 (1998). As an outbred species, marmosets exhibit a very broad immunologic repertoire against myelin antigens, which is similar to humans. In addition to whole myelin and MOG, susceptibility to myelin basic protein (MBP), MBP-derived peptides, and proteolipid protein (PLP) has been demonstrated. Genain et al., Immunol. Reviews 183:159-172 (2001). Diverse epitope recognition and T cell receptor β chain utilization are seen in the encephalitogenic repertoires against myelin proteins. Genain et al., J. Clin. Invest. 94:1339-1345 (1994); Uccelli et al., Eur. J. Immunol. 31:474-479 (2001); Villoslada et al., Eur. J. Immunol. 31:2942-2950 (2001); and Mesleh et al., Neurobiol Dis 9:160-172 (2002). C. jacchus are unique primates for studies of autoimmunity because these monkeys are born as naturally occurring bone marrow chimeras. While sibling pairs or triplets are genetically distinct, they share, and are tolerant to, each other's bone marrow-derived cell populations, which permits adoptive transfer of T cell clones. Genain et al., J. Clin. Invest. 94:1339-1345 (1994); Villoslada et al., Eur. J. Immunol. 31:2942-2950 (2001); and Watkins et al., Journal of Immunology 144:3726-3735 (1990).

The MS-like lesion in C. jacchus is mediated by a complex interplay between cellular and humoral responses to myelin. MOG has been shown to be a target for demyelinating antibodies. Genain et al., J. Clin. Invest. 96:2966-2974 (1995). Importantly, pathogenicity of MOG-specific autoantibodies has also been demonstrated in selected cases of human MS. Genain et al., Nat Med 5:170-175 (1999). C. jacchus shares a very high degree of homology with humans for myelin and immune system genes. The recent cloning of MOG-specific marmoset immunoglobulin genes has revealed similarity of gene usage and epitope recognition between marmosets and humans. von Büdingen et al., Immunogenetics 53:557-563 (2001) and von Büdingen et al., Proc. Natl. Acad. Sci. USA 99:8207-8212 (2002).

Human herpesvirus (HHV)6 has been implicated in the etiology of multiple sclerosis (MS), based on detection of HHV6 DNA in MS plaques and serum, presence of anti-HHV6 reactivity in MS-affected individuals, and reports of encephalitis or encephalomyelitis associated with this virus. Epidemiological studies indeed suggest that viruses or other environmental factors may trigger MS or influence its course. As for other viruses, however, evidence for a direct link of causality between HHV6-A and disease pathogenesis has been lacking.

The two HHV6 variants (HHV6-A and HHV6-B) show capability to infect a wide range of human and primate host cells. HHV6-B causes exanthema subitum, a mostly benign febrile illness in children. A cellular receptor for HHV6 has been recognized as the membrane cofactor protein (CD46). CD46 is a ubiquitous receptor promiscuous to other microbes and herpesviruses including measles, and belongs to a family of complement receptor proteins. High levels of soluble CD46 are observed at early stages of MS—a finding also interpreted as evidence for a role of HHV6 infection in relapses.

HHV6 is a herpesvirus that possesses a 159 kbp to 170 kbp long genome with 7 gene blocks common to all Herpesviridae, a group of genes found only in β-herpesviruses (ORFs U2 to U14) and genes specific to the Roseolavirus genus (ORFs U15 to U25). Three genes, U22, U83 and U94, are specific for HHV6 (not HHV7). HHV6-B contains 119 ORFs in comparison with 110 for HHV6-A. Dockrell, J Med Microbiol 52:5-18 (2003). Despite being very similar, the two HHV6 variants have very different cell tropism and disease manifestations, which support the concept that they are different herpesviruses.

HHV6-B causes exanthema subitum in children, or initial exposure may be asymptomatic. Practically all individuals get infected prior to age 2 (Caserta et al., J Pediatr 145:478-84 (2004) and Zerr et al., N Engl J Med 352:768-76 (2005)). HHV6-B is found in a wide variety of tissues, including lymphoid organs, brain, serum and salivary glands. Ablashi et al., J Virol Methods 21:29-48. (1988); Levy et al., Lancet 335:1047-1050 (1990); Levy et al., Virology 178:113-121 (1990) and Lusso et al., Baillieres Clin Haematol 8:201-23 (1995)).

HHV6-A has a particular tropism for the CNS and skin. This variant has so far rarely been isolated or detected in children with primary HHV6 infection, and is not clearly associated with any infectious illness in healthy populations. HHV6 persists in latent or replicative states throughout life, and actively replicates in salivary glands (variant B). Secondary infection by HHV6 is usually silent except in immuno-compromized patients. Dockrell, J Med Microbiol 52:5-18 (2003) and Campadelli-Fiume et al., Emerg Infect Dis 5:353-366 (1999). Antibodies against HHV6-A are found in most of the general population, and steadily persist through life before declining in older subjects. Levy, Lancet 349:558-563 (1997).

The CD46 cellular HHV6 receptor (Santoro et al., Cell 99:817-827 (1999)) is expressed ubiquitously, including in CNS, but only in humans and certain higher mammals and primates, which explains the narrow range of species that can be infected with this virus. CD46 binds to the C3b and C4b proteins and inactivates the complement system. Thus, one of its presumed functions is to protect the cells from self-lysis by complement. HHV6 is capable of infecting CD4+, CD8+, NK and γδ T cells, B cells, macrophages, dendritic cells, fibroblasts, epithelial cells and a variety of lymphoid or CNS-derived cell lines. Dockrell, J Med Microbiol 52:5-18 (2003); Campadelli-Fiume et al., Emerg Infect Dis 5:353-366 (1999); and Levy, Lancet 349:558-563 (1997). Both variants infect primary fetal astrocytes, but HHV6-A appears to have a greater neurotropism in vivo. Hall et al., Clin Infect Dis 26:132-137 (1998). Infection in vitro by HHV6 is monophasic and generally followed by decreased cell proliferation and/or cell death. Grivel et al., J Virol 77:8280-9 (2003); Opsahl et al., Brain 128:516-27 (2005); and Smith, et al., J Virol 79:2807-13 (2005). In vivo, HHV6 induces CD4 T cell depletion, as shown in a SCID mouse model implanted with human fetal thymus and liver (Gobbi et al., J Exp Med 189:1953-1960 (1999)), and may contribute to HIV-associated immunosuppression. It is also clear that HHV6 infection interferes with other viruses, including EBV, cytomegalovirus (CMV), and human immunodeficiency virus (HIV) for which either enhancing or suppressing effects have been described. Levy, Lancet 349:558-563 (1997).

The CD46 receptor is shared by a number of pathogens including measles virus, and signaling through this molecule is one of the most potent mechanisms of T cell stimulation and activation. Several isoforms of CD46 that differ by their cytoplasmic domains are expressed in humans, and engagement of these 2 classes of CD46 receptors appears to have opposite consequences on the polarization of the immune response towards Th1 or Th2 phenotypes (Marie et al., Nat Immunol 3:659-66 (2002); Russell, Tissue Antigens, 64:111-8 (2004); and Riley-Vargas et al., Trends Immunol 25:496-503 (2004)).

In vivo, HHV6 induces CD4+ T cell depletion, as shown in a SCID mouse model implanted with human fetal thymus and liver (Gobbi et al., J Exp Med 189:1953-60 (1999) and Gobbi et al., J Virol 74:8726-31 (2000)), and may contribute to HIV-associated immunosuppression (Lusso et al., Immunol Today 16:67-71 (1995b)). It is also clear that HHV6 infection interferes with other viruses, including EBV, cytomegalovirus (CMV), and human immunodeficiency virus (HIV) for which either enhancing or suppressing effects have been described (Levy, Lancet 349:558-63 (1997) and Ablashi et al., J Virol Methods 21:29-48 (1988)). A number of attempts have been made to create models of infection with HHV6 in primates (macaques and chimpanzees), which as in the SCID mouse model primarily support the concept that HHV6-A acts as a cofactor in the simian acquired immunodeficiency syndrome (Lusso et al., J Virol 64:2751-8 (1990) and Lusso et al., AIDS Res Hum Retroviruses 10:181-7 (1994)).

Because >95% of the general population is exposed to the virus during infancy, it is difficult to envision HHV6 infection as a sole cause for MS prevalence (approximately 1:1,000 for Caucasions in the United States) in the absence of additional factors of pathogenesis. One possible scenario that has been proposed to explain the association of MS with viral exposure is that primary infection triggers a silent immune attack on the central nervous system (CNS), which is followed over time by development of CNS-directed autoimmunity. In favor of this hypothesis are findings that the risk of developing MS appears to be acquired early in life, and follows migration patterns to and from geographical areas of low/high prevalence if individuals are moved during their childhood. MS epidemics have also been observed after novel exposure of previously isolated insular populations to foreign environmental factors. Yet, there is still no direct evidence of an association between any viral exposures and common forms of MS.

Of particular relevance to MS are recent observations that both HHV6 variants show capability to modulate T cell inflammatory responses towards Th1 (pro-inflammatory) phenotypes. Mayne et al., J Virol 75:11641-11650 (2001). In addition, a consequence of infection of endothelial cells by HHV6-A appears to be an increase in vascular endothelium permeability. Caruso et al., J Med Virol 67:528-533 (2002). Thus, in keeping with the concept of heterogeneity in MS pathophysiology, it is possible that an association between MS and HHV6 exists for certain clinical or neuropathological subtypes that are yet to be identified. It is, however, premature to conclude that this is sufficient evidence that this virus causes MS, which is commonly regarded as a disease with generalized autoimmune dysregulation; findings of viral DNA, antibody reactivity, or even association with viral infections may indeed represent a consequence of the disease rather than its cause.

An association between HHV6-A and MS was recently suggested by findings of HHV6-B DNA sequences in diseased oligodendrocytes within MS plaques (Challoner et al., Proc. Natl. Acad. Sci. 92:7440-7444 (1995); Opsahl et al., Brain 128:516-27 (2005). These observation however, has not been confirmed by subsequent attempts (Coates et al., Nat Med 4:537-8 (1998)), and could not be formally confirmed by immunohistochemistry. HHV6 DNA has also been found in the brain of normal subjects and in Alzheimer's disease (Luppi et al., J Med Virol 47:105-11 (1995); Lin et al., J Pathol 197:395-402 (2002)). Thus, detection of viral sequences in the CNS is not sufficient for proof of pathogenicity. Serologic studies have reported elevated titers of anti-HHV6-Antibodies in patients with relapsing remitting MS compared to controls (Ablashi et al., Mult Scler 4:490-6 (1998); Sola et al., J Neurol Neurosurg Psychiatry 56:917-9(1993); Soldan et al., Nature Medicine 3:1394-7 (1997)). However, a large number of subsequent studies that examined IgG/IgM reactivity in serum and/or CSF, presence of HHV6 DNA or viral transcripts in serum, CSF and brain, peripheral T cell proliferative responses to HHV6, or virus recovery in culture have not unequivocally confirmed these results. The following reviews provide detailed discussions of the numerous HHV6 association studies that have been performed (Ablashi et al., J Virol Methods 21:29-48 (1988); Ablashi et al., Mult Scler 4:490-6 (1998); Krueger et al., Pathol Res Pract 185:915-29 (1989); Enbom, Apmis 109:401-11 (2001); Moore et al., Acta Neurol Scand 106:63-83 (2002); Krueger et al., Intervirology 46:257-69 (2003); DeRanieri et al., J Sch Nurs 20:69-75 (2004); Dewhurst, Herpes 11 Suppl 2:105A-111A (2004); Fotheringham et al., Herpes 12:4-9 (2005)).

More compelling for an association between HHV6-A and certain forms of CNS demyelination which possibly represent extremes of the spectrum of MS presentations are numerous case reports of encephalomyelitis, and acute and chronic myelitis where a clear relationship between the infection and CNS disease was strongly suggested (Carrigan et al., Neurology 47:145-148 (1996); Mackenzie et al., Neurology 45:2015-7 (1995); McCullers et al., Clin Infect Dis 21:571-6 (1995); Novoa et al., J Med Virol 52:301-8 (1997); Portolani et al., J Med Virol 65:133-7 (2001); Portolani et al., Minerva Pediatr 54:459-64 (2002); Singh et al., Transplantation, 69:2474-9 (2000); Moore et al., Acta Neurol Scand 106:63-83 (2002); Dockrell, J Med Microbiol 52:5-18 (2003); Campadelli-Fiume et al., Emerg Infect Dis 5:353-66 (1999); Gilden et al., Multiple Sclerosis 2:179-183 (1996); Kleinschmidt-DeMasters et al., Brain Pathol 11:440-51 (2001); Ward, Curr Opin Infect Dis 18:247-52 (2005).

In addition to MS and encephalomyelitis, and association with HHV6 exposure and HHV6 reactivity has also been claimed for chronic fatigue syndrome and narcoplepsy. Chronic fatigue syndrome (CFS) is an incapacitating disease of adult of all ages, which shares certain clinical features with MS (the fatigue) and is also likely immune-mediated. Similar to MS, studies of antibody reactivity have been inconsistent in proving a relationship between CFS and HHV6 (Enbom, Apmis 109:401-11 (2001); Ablashi et al., J Clin Virol 16:179-91 (2000); Wallace et al., Clin Diagn Lab Immunol 6:216-23 (1999); Nicolson et al., Apmis 111:557-66 (2003)).

Experimental systems are needed to understand causal relationships between HHV6 infection and the occurrence of CNS inflammatory conditions that mimic human MS. Only the availability of such models will permit studies of causal and time-dependent relationships between infection and CNS disease in a controlled fashion. Thus, there remains a need in the art for experimental systems that permit longitudinal studies following HHV6 exposure in order to characterize the role of this virus in autoimmune CNS demyelination and animal model systems suitable of identifying and characterizing efficacious therapeutics and treatment regimens effective in ameliorating or decreasing the severity of this autoimmune disease.

### SUMMARY OF THE INVENTION

The present invention addresses these and other related needs by providing, inter alia, non-human animal model systems for viral pathogenesis of neurodegeneration, autoimmune demyelination, and diabetes. Such animal model systems may be suitably employed for the study of multiple sclerosis (MS) and for the identification and characterization of candidate therapeutic compounds and compositions for the treatment of MS.

Animal model systems according to the present invention are correlative of MS disease in humans and, thus, will find a wide range of utilities. Such animal model systems will, for example: (1) provide an opportunity to identify the factors controlling the pathogenesis of CNS autoimmunity following exposure to HHV6; (2) provide a suitable system for identifying and characterizing potentially efficacious therapeutic agents for the treatment of MS disease; (3) provide a suitable system for performing similar investigations and therapeutic testing for additional or alternative neurodegenerative and autoimmune, immune-mediated or infectious and post-infectious human conditions; (4) permit the discovery of biomarkers for the detection of MS; and (5) lead to the development of strategies and/or treatment regimens to remedy HHV6 induced CNS pathology.

Within certain embodiments, the non-human animal is a non-human primate wherein the primate is infected with a herpesvirus. Typically, non-human primates suitably infected with a herpesvirus according to the present invention include monkeys and are selected from the group consisting of a marmoset, a New World monkey, and an Old World monkey, wherein the primate is susceptible to infection with said herpesvirus.

Exemplified herein are non-human primate animal model systems wherein a marmoset (C. jacchus) is infected with a herpesvirus. More specifically, presented herein are non-human animal model systems of MS disease that are based upon the in vivo infection of a non-human animal with HHV6. An exemplary animal model of HHV6-induced CNS demyelination has been created in the common marmoset C jacchus, a New World non-human primate that develops spontaneous autoimmunity and is also used in studies of experimental allergic encephalomyelitis.

Captive marmosets are naïve to HHV6, and express a CD46 that is homologous to human CD46, which affords the opportunity, as presented herein, to model the events following initial and subsequent exposures, and to study the consequences of infection. CNS autoimmune demyelination appears associated with repeated exposures of adult marmosets to HHV6-A.

Thus, within certain embodiments are provided C. jacchus marmosets that are infected with a herpes virus, exemplified by one or more HHV6 variants. While infection with HHV6 is monophasic and rapidly lethal to the cells in vitro (HHV6 is capable of inducing apoptosis in CNS glial cells), it is demonstrated herein that a CNS demyelinating disorder follows infection of naïve adult marmosets with HHV6-A. In some instances, it is further demonstrated that certain animals proceed to develop lesions of the gray matter, especially the basal ganglia, and marked brain atrophy. Without wishing to be limited to any particular mode of action, it is believed that this CNS disease is associated with the appearance of T cell reactivity to myelin antigens.

A wide variety of herpesviruses may be suitably employed in the non-human primate animal model systems disclosed herein. Particularly suitable are those herpesviruses that are capable of specifically binding to a CD46 receptor. Exemplified herein are non-human primate animal model systems infected with a herpesvirus selected from the group consisting of HHV6-A and HHV6-B.

Depending upon the precise application contemplated, non-human primates may be infected by a single exposure to a single herpesvirus variant whereby infection of the non-human primate with the herpesvirus triggers and/or increases the severity of a central nervous system inflammatory disease. Alternatively, other applications may require that non-human primates are infected by more than one exposure to a single herpesvirus variant wherein more than one exposure of the non-human primate to said herpesvirus triggers and/or increases the severity of a central nervous system inflammatory disease. Further provided are non-human primate animal model systems wherein a primate is infected with one or more exposure to more than one herpesvirus variant. Particularly suitable to the non-human primate animal model systems presented herein are herpesvirus variants selected from the group consisting of HHV6-A and HHV6-B.

Non-human primate animal model systems of the present invention are suitably employed for studying disease mechanisms and for identifying and characterizing candidate therapeutics for a number of diseases of the central nervous system, in particular inflammatory and demyelinating diseases of the central nervous system. Exemplified herein are non-human primate animal model systems of multiple sclerosis. Within relates aspects, exposures of a non-human primate with one or more herpesvirus variant may further trigger and/or increases the severity of other inflammatory diseases or malignancies of the central or peripheral nervous system and neuromuscular junction.

For example, exposure of a non-human primate to one or more herpesvirus may trigger and/or increase the severity of a disease selected from the group consisting of a paraneoplastic syndrome and cerebellar degeneration, limbic encephalitis, opsoclonus myoclonus, subacute sclerosing panencephalitis (SSPE), progressive multifocal leukoencephalopathy (PML) and other diffuse or focal leukodystrophies (early and late onset), acute and chronic polyneurpathies and polyradiculopathies, acute disseminated encephalomyelitis, myopathy, myasthenia gravis, Guillain Barre, miller-Fisher syndrome, Eaton Lambert syndrome, CNS vasculitis, sarcoidosis and neurosarcoid, Rasmussen's disease, paraneoplastic sensory neuropathy, CNS lymphoma, high and low grade oligodendroglioma and glioblastoma, glioblastoma multiformis, optic nerve glioma and meningioma, ependymoma, and medulloblastoma.

Alternative aspects of the present invention provide that exposure of a non-human primate to one or more herpesvirus may trigger and/or increase the severity of a neurological disorder comprising an inflammatory component selected from the group consisting of narcolepsy, chronic fatigue syndrome, stiff man syndrome, and childhood autism.

Still further aspects of the present invention provide that exposure of a non-human primate to one or more herpesvirus may trigger and/or increase the severity of an inflammatory disease and/or autoimmune disorder selected from the group consisting of diabetes, arthritis, anemia, lupus, pemphigus, thyroiditis, glomerular or intersticial nephritis, cardiomyopathy, myositis, dermatomyositis, hepatitis, and ulcerative colitis.

Yet other aspects of the present invention provide non-human primate animal model systems that are suitable for the identification of factors mediating the direct toxicity of one or more herpesvirus and a cell type selected from the group consisting of an oligodendrocyte, an astrocyte, and a brain cell.

Other embodiments of the invention disclosed herein provide non-human animal model systems for the study of brain or spinal cord atrophy and degeneration in a disease affecting basal ganglia and gray matter wherein the disease is selected from the group consisting of Alzheimer's disease, Parkinson's disease, Lewy body disease, Lafora disease, chorea and athetosis, Huntington's disease, and amyotrophic lateral sclerosis (Lou Gherig's disease).

Further embodiments provide non-human animal model systems for the study of the interaction between a virus and a primate immune system wherein the primate is selected from the group consisting of a marmoset, a New World monkey, and an Old World monkey. Certain aspects of such embodiments provide non-human animal model systems for studying the interactions between virus pairs wherein said virus pairs are selected from the group consisting of: (a) HHV6-A and HHV6-B; (b) HHV6 and CMV; (c) HHV6 and EBV; (d) HHV6 and VZV; (e) HHV6 and HHV8; (f) HHV6 and HIV; and (g) HHV6 and HTLV.

Other embodiments of the present invention provide experimental systems for studying the potential of a candidate compound for reducing the severity of a disease, wherein the experimental systems comprise a herpesvirus infected non-human animal; wherein the disease is selected from the group consisting of a demyelinating disease, a neurodegenerative disease, and multiple sclerosis; and wherein reduction in the severity of the disease is determined by measuring an inhibition of viral replication and/or transcription. Certain aspects of the experimental systems provided herein comprise a mammal selected from the group consisting of a monkey, a wild-type mouse, an EAE mouse, and a CD46 transgenic mouse; wherein said experimental system permits the testing of soluble CD46 (complement receptor) as a therapeutic agent.

Related aspects of the present invention provide experimental non-human animal model systems for the study of potential vaccine therapeutics for reducing the severity of a disease selected from the group consisting of an autoimmune and/or neurodegenerative disease such as multiple sclerosis. Such experimental systems typically comprise a herpesvirus infected non-human animal such as a rodent or non-human primate. Exemplified herein are experimental non-human animal model systems wherein the herpesvirus is, for example, HHV6-A and/or HHV6-B.

Still further related aspects include experimental systems for the identification of genes responsible for the development of an autoimmune and/or neurodegenerative disease following exposure to a herpesvirus, wherein the experimental system employs a technique selected from the group consisting of a gene expression array, proteomics, metabonomics, and metabolonics.

Yet other related aspects include experimental systems for the identification of genes responsible for the development of a detrimental autoantibody response that may lead to autoimmune and/or neurodegenerative disease following exposure to a herpesvirus, wherein the experimental system employs a technique selected from the group consisting of a gene expression array, proteomics, metabonomics, and metabolonics.

Other related aspects include experimental systems for the identification of genes responsible for the development of a beneficial autoantibody response such as, for example, a neutralizing antibody response against a herpesvirus, wherein the beneficial autoantibody response prevents, or substantially reduces, the development of an autoimmune and/or neurodegenerative disease following exposure to a herpesvirus. Such experimental systems typically employ a technique selected from the group consisting of a gene expression array, proteomics, metabonomics, and metabolonics.

Within other embodiments of the present invention are provided transgenic animal model systems, such as mouse, zebrafish, drosophila, and nematode animal model systems, comprising a transgene encoding CD46 and a herpesvirus. Exemplified herein is a transgenic mouse animal model system wherein the transgenic mouse comprises a transgene encoding CD46, wherein the transgenic mouse is infected with a herpesvirus, and wherein the herpesvirus is typically selected from the group consisting of HHV6-A and HHV6-B. Within certain aspects of these embodiments, the transgene encoding CD46 is ubiquitously expressed in vivo. Within alternative aspects, the transgene encoding CD46 is expressed in vivo in a tissue selected from the group consisting of brain, spinal cord, and peripheral nerve.

Transgenic mouse animal model systems presented herein may be achieved by a single exposure of the CD46 transgenic mouse to a herpesvirus wherein such viral exposure triggers and/or increases the severity of a central nervous system inflammatory disease. Within alternative aspects, more than one exposure of the transgenic mouse to a herpesvirus is required to trigger and/or increase the severity of a central nervous system inflammatory disease. Within yet further aspects of the present invention, the CD46 transgenic mouse is exposed to a combination of two or more viruses such as, for example (a) HHV6-A and HHV6-B; (b) HHV6 and CMV; (c) HHV6 and EBV; (d) HHV6 and VZV; (e) HHV6 and HHV8; (f) HHV6 and HIV; and (g) HHV6 and HTLV.

Transgenic mouse animal model systems disclosed herein are suitably employed for studying the potential of a candidate compound for reducing the severity of a disease of the central or peripheral nervous system such as, for example, a nervous system inflammatory disease. Typically, exposure of a CD46 transgenic mouse with one or more herpesvirus, as described herein, triggers and/or increases the severity of an inflammatory disease and/or autoimmune disorder selected from the group consisting of multiple sclerosis, diabetes, arthritis, anemia, lupus, pemphigus, thyroiditis, glomerular or interstitial nephritis, cardiomyopathy, myositis, dermatomyositis, hepatitis, and ulcerative colitis. Such herpesvirus infected CD46 transgenic animal model systems are suitable for the identification of factors mediating the direct toxicity of the herpesvirus towards a cell type such as, for example, a cell type selected from the group consisting of an oligodendrocyte, an astrocyte, and a brain cell. Exemplary factors include, without limitation, cells of the immune system such as CD4+ T-cells and CD8+ T-cells.

Other embodiments of the present invention provide compositions comprising a CD46 variant selected from the group consisting of (a) a soluble CD46, (b) a cell associated CD46, and (c) an artificial delivery system associated CD46; wherein the composition is effective in reducing the severity of a disease selected from the group consisting of multiple sclerosis and/or other autoimmune and immune-mediated inflammatory diseases of the brain or other target organs; wherein the CD46 is produced in recombinant form, as a full-length polypeptide or as a truncated variant; and wherein the artificial delivery system is either a liposome or a vesicle. Within certain aspects, such compositions are effective in the treatment of a neurodegenerative disorder and/or a tumor.

Still further embodiments of the present invention provide methods for detecting a patient at risk for developing a disease such as, for example, multiple sclerosis and/or other autoimmune and immune-mediated inflammatory diseases of the brain or other target organs. Within certain aspects, such methods comprise the steps of: (1) isolating from the patient a biological sample suspected of comprising an antibody that specifically binds to human CD46; (2) contacting the biological sample with a cell expressing human CD46 or a variant thereof for such a time and under such conditions as required to achieve a first complex comprising the antibody that specifically binds to human CD46 and the cell expressing human CD46; (3) contacting said complex with a secondary anti-human antibody wherein said secondary antibody comprises a detectable tag for such a time and under such conditions as required to achieve a second complex comprising said secondary anti-human antibody specifically bound to said first complex; and (4) detecting said detectable tag on said bound secondary antibody. Typically, the detectable tag on the secondary antibody is detected by means of fluorescence activated cell sorting analysis or other method where a detection tag is used to reveal the presence of the secondary antibody. Detectable tags may be fluorescent tags or may be radioisotopes. Within certain aspects, methods according to these embodiments may be suitably employed for identifying a patient wherein an active destructive process is linked to or concomitant with herpesvirus replication, including HHV6 replication, and activity is ongoing. By such methods, early treatment regimens may be initiated in the patient whereby full development of a disease such as multiple sclerosis, chronic fatigue syndrome, and other related disorder is prevented.

Related embodiments of the present invention provide methods to evaluate in a patient, such as a human patient, the existence of antibodies or cellular responses that result in neutralization of herpesvirus-mediated infections, such as HHV6-mediated infections. Similar methods are provided that permit the evaluation of such patients for failure to produce an antibody and/or T cell response resulting in early or delayed organ-specific autoimmunity, including multiple sclerosis and diabetes. By these methods, antibodies, such as neutralizing antibodies, or cellular responses are detected and correlated with the risk of a patient developing a disease of the central nervous system, such as multiple sclerosis and/or the risk of a patient developing an autoimmune disorder selected from the group consisting of diabetes, arthritis, anemia, lupus, pemphigus, thyroiditis, glomerular or interstitial nephritis, cardiomyopathy, myositis, dermatomyositis, hepatitis, and ulcerative colitis.

Alternative related aspects of these embodiments include methods for identifying a compound effective in reducing the severity of herpesvirus-mediated toxicity in a cell within a patient sample, wherein such methods comprise the steps of (a) administering to a non-human animal model system, as described herein, a candidate compound and (b) determining whether the herpesvirus-mediated toxicity is reduced in severity. Typically, such herpesvirus-mediated toxicity is correlative of a neurodegenerative disease selected from the group consisting of multiple sclerosis, Parkinson's disease, Alzheimer's disease, and cerebellar degeneration. Exemplary cells within a patient sample include neurons and cells within a patient's serum, blood, cerebral spinal fluid (CSF), and/or other patient samples. Measurements of cellular toxicity include toxicity, lytic effect, cytokine-mediated death, apoptosis.

Also provided are methods for evaluating the therapeutic value of a compound or other intervention that antagonizes the development of detrimental autoantibodies the generation of which is induced by exposure to a herpesvirus such as, for example, HHV6-A and/or HHV6-B. Related methods are provided for evaluating the therapeutic value of a compound or other intervention that favors the development of a beneficial autoantibody. Additional methods are provided for evaluating the therapeutic value of a compound or intervention that alters the immune system via its cellular responses such that detrimental autoantibodies are antagonized or beneficial autoantibodies are agonized.

The present invention also provides, in other embodiments, methods for detecting in a patient the risk of infection with a ubiquitous virus in a disease state such as multiple sclerosis and/or another autoimmune disorder wherein the patient is susceptible to immunosuppression, transplant, AIDS, and/or other immunodeficiency.

### BRIEF DESCRIPTION OF THE FIGURES

FIGS. 1A-1C are Luxol fast blue/periodic acid Schiff (LFB/PAS) stained tissue sections depicting the neuropathology of C. jacchus EAE. FIG. 1A depicts large perivascular infiltrates in the lateral and posterior spinal cord funiculi (acute EAE). FIG. 1B is a low-power view of brain perivascular infiltrates in periventricular white matter. FIG. 1C is a high-power magnification of the same lesion illustrating mononuclear cell and macrophage infiltration with prominent demyelination (LFB/PAS).

FIGS. 2A-2C are tissue sections comparing C. jacchus EAE and human MS. FIG. 2A depicts acute C. jacchus EAE primary demyelination with preservation of axons (Ax), macrophage infiltration (nucleus at Mac, top right) and astrogliosis (gl). Typical morphologic changes of myelin dissolution and vesiculation are visible (*). FIG. 2B depicts an acute human MS lesion (biopsy) showing the same characteristic pattern of myelin vesiculation around an axon (Ax). A macrophage nucleus is visible at the top right. FIG. 2C depicts chronic C. jacchus EAE illustrating intense gliosis (gl) and thin compact myelin around axons (Ax) indicative of remyelination. For comparison a normally myelinated axon (thick myelin) is shown in the upper right hand corner (*).

FIGS. 3A-3D depict the in vitro infection of marmoset peripheral blood mononuclear cells (PBMC) with HHV6. FIG. 3A is a photograph of a DNA gel depicting HHV6 DNA amplified by nested PCR (expected fragment size 258 bp). In lane 1 is DNA from a marmoset PBMC infected with HHV6-A, 10 days after infection. In lane 2 is DNA from an uninfected T cell line HSB2. In lanes 3 and 8 are DNA from HSB2 infected with HHV6-A. In lane 4 is DNA from uninfected T cell line MOLT3. In lanes 5 and 9 are DNA from MOLT3 infected with HHV6-B. In lane 6 is a template only control. In lane 7 is DNA from marmoset PBMC infected with HHV6-B. FIGS. 3B-D depict cells that were immunofluorescence (IFA) stained for HHV6 nuclear antigen p41. FIG. 3B depicts HHV6-A-infected marmoset PBMC. FIG. 3C depicts HHV6-A-infected HSB2 cells. FIG. 3D depicts uninfected marmoset PBMC.

FIG. 4 depicts the clinical course (neurological signs) in seven (7) animals studied using a marmoset EAE grading scale (0-45). Villoslada et al., J. Exp. Med. 191:1799-1806 (2000).

FIG. 5 depicts coronal MRI contiguous sections of the entire brain from animal 190-94 (infected with HHV6-A) in vivo, immediately prior to enthanasia. Sections are numbered 1 to 15 from rostral to caudal direction. Note hypointense T2-weighted signal in left striatum on section no. 3 (white arrow), and ill-defined, irregular lesion adjacent ot the 4^{th} ventricle in section no. 12 (black arrow), representing the demyelinating lesion shown in FIG. 7A.

FIG. 6 depicts coronal MRI contiguous sections of the entire brain from animal U031-00 (infected with HHV6-A) in vivo, immediately prior to euthanasia. Sections are numbered 1 to 15 rostral to caudal. Note the prominent sulci and ventricles (white arrows), with striking lateralization and asymmetry reflected in enlargement of the cerebrospinal and ventricular spaces on the left side of the brain involving the temporal and occipital lobe (black arrows). Regional atrophy (black arrows) is evident on sections no. 6, 9 and 10, which can be compared to equivalent sections shown in FIG. 5.

FIG. 7A depicts demyelinating inflammatory infiltrate in the brain stem of animal 190-94 (luxol fast blue). FIG. 7B depicts staining for early nuclear antigen p41/p38 demonstrating viral persistence/replication within lesions.

FIG. 8 are graphs of flow cytometry data showing serum reactivity to HHV6-A⁺-HSB2 cells in animal 190-94. The upper-left panel depicts staining for isotype control. The upper-right panel depicts staining for CD46. The middle-left panel depicts control anti-monkey IgG antibody. The middle-right panel depicts naïve serum (day 0). The bottom-left panel depicts serum after the first inoculation (day 35). The bottom-right panel depicts serum at euthanasia after the second inoculation. The open trace represents the negative signal obtained with anti-monkey IgG-FITC.

FIG. 9 depicts the gel electrophoretic detection of HHV6-B DNA in PBMC. In lane 1 is DNA from an HHV6-B-infected animal 7 weeks after inoculation. In lane 2 is DNA from an HHV6-A-infected animal 7 weeks after inoculation. Lanes 3-6 are negative controls. Lanes 7 and 8 are DNA from control HHV6-A and B infected lines.

FIGS. 10A-10B are charts depicting T cell proliferative responses against MBP, MOG (extracellular domain), and a mixture of 20 mer overlapping MOG peptides in animal 190-94 and 125-. Data are obtained from PBMC at euthanasia.

FIG. 11 depicts the influence of measles virus sensitization on murine EAE.

FIG. 12 depicts an example of relapsing marmoset EAE with characteristic neuropathological features at each stage.

FIGS. 13A-13B depict presence of hyper-intense T2-weighted lesions corresponding to perivascular infiltrates with inflammation and demyelination in animals receiving live HHV6-A virus twice. FIG. 13A depicts hyper-intense T2 lesion in the animals' brain stem, adjacent to IV^{th} ventricle. FIG. 13B depicts apoptotic cells observed within lesions (TUNEL) of brain sections of HHV6-A-infected animals.

FIGS. 14A-14C depict the effect of a specific pro-apoptotic effect of HHV6 variants on human oligodendrocytoma cell line TC620. FIGS. 14A and 14B depict the increase of apoptosis (R4) and decrease of live cells (R2) in TC620 cells co-incubated with HHV6-A-infected cell line (A) compared to the non-infected cell line (background, B). FIG. 14C depicts the percent increase of oligodendrocyte apoptosis observed after co-incubation with HHV6-A and HHV6-B infected cell lines.

FIG. 15A depicts the clinical course for animals inoculated with HHV6-A and HHV6-B; FIGS. 15B and 15C depict measurements of peripheral T cell immune reactivity (PBMC) to phytohemagglutinin (PHA), myelin/oligodendrocyte glycoprotein (MOG), and myelin basic protein (MBP) in serial blood samples of the animals.

FIGS. 16A-16C depict representative flow cytometry data showing heterogeneous staining (low to high) for CD25 (FITC) in a healthy control (FIG. 16A), a patient with MS treated with IFN-β alone (FIG. 16B), and the patient receiving natalizumab+IFN-β that developed PML (FIG. 16C).

FIGS. 17A-17C depicts neuropathologic findings in animal U076-03, inoculated as animal 190-94 twice with live HHV6-A. FIG. 17A is a low power view showing a large inflammatory infiltrate in subcortical white matter. FIG. 17B is a detail of the infiltrate showing intense infiltration by mononuclear cells and macrophages (arrowheads) around blood vessels, and numerous areas of myelin vacuolation and breakdown (arrows) typical of marmoset EAE and acute MS lesions (H&E; GM: gray matter; WM: white matter). FIG. 17C is Luxol fast blue/PAS staining of a peri-ventricular inflammatory infiltrate, showing prominent demyelination and macrophage activity.

FIG. 18 depicts immunohistochemical staining showing staining of oligodendrocytes in periventricular white matter (corpus callosum) devoid of lesions. This demonstrates that viral replication took place in brain areas devoid of inflammatory demyelinating infiltrates.

FIG. 19A depicts staining of replicating HHV6 virus (p41) at the site of cellular lesions and FIG. 19B depicts staining of oligodendrocytes (CNPase) at the location of replicating HHV6 virus.

FIGS. 20A-20H depict all lymphocyte subsets analyzed in patients treated with natalizumab+IFN-β, patients treated with NMO, and patients with MS treated with conventional DMT. Circles are Natalizumab+Avonex (IFN-β); triangles pointing upward are NMO (middle); and triangles pointing down are MS+disease modifying therapies approved by FDA (IFN, Copaxone, called collectively DMT). FIG. 20A depicts the ratio of CD19⁺/CD3⁺ counts; FIG. 20B depicts absolute counts of activated T regulatory cells (CD4+CD25⁺); FIG. 20C depicts total white blood cell counts; FIG. 20D depicts total lymphocyte counts; FIG. 20E depicts total helper T cells (CD3+CD4+) ratio; FIG. 20F depicts total CD8+ suppressor T cells (CD3+CD8+); FIG. 20G depicts total B cells (CD19+); and FIG. 20H depicts the percentage of CD19⁺ B cell counts relative to total lymphocyte counts.

FIGS. 21A-21C depict total lymphocyte (FIG. 21A), CD19⁺ cells (FIG. 21.B), and CD3⁺ cells in five patients following treatement with natalizumab (Tysabri), three patients with neuromyelitis optica (NMO) treated with steroids and plasma exchange, and five patients with relapsing remitting multiple sclerosis (MS), treated with approved disease modifying therapies (interferon-β 1-b, interferon-β 1-a or copolymer 1).

FIG. 22 depicts a time course for the appearance of weight loss and elevated blood sugar values in animal 50-01, inoculated×2 with HHV6-B variant. Animal 50-01, unlike those inoculated with HHV6-A variant, did not develop any significant neurological deficit. The animal also had >1,000 mg/dl in a urine sample at the time it was diagnosed with diabetes and experienced abrupt weight loss (˜27% initial weight, around 210 days after initial inoculation, arrow 1). * denotes a blood sugar measurement done as a routine health check 2 years prior to the beginning of the current experiment. This value, and that around day 210 are within normal limits (Yarbrough et al., Lab Animal Science, (1984).

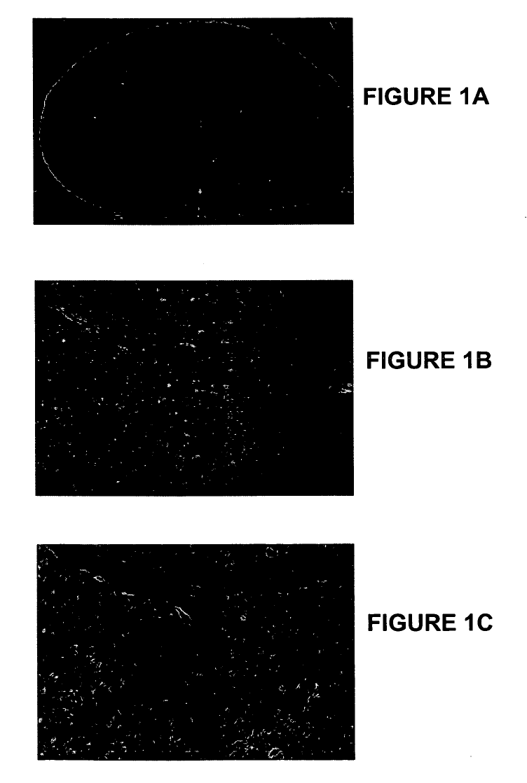

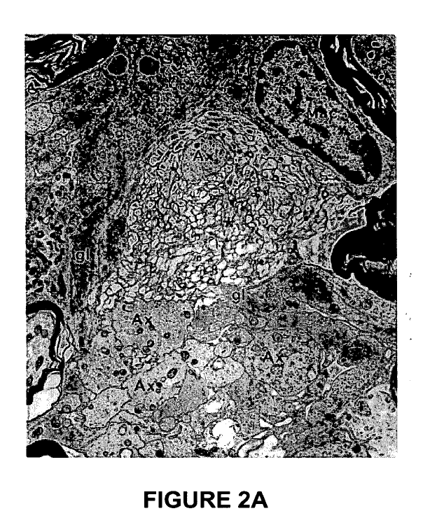

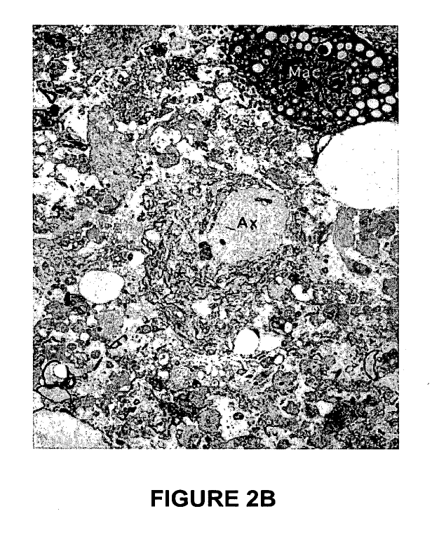

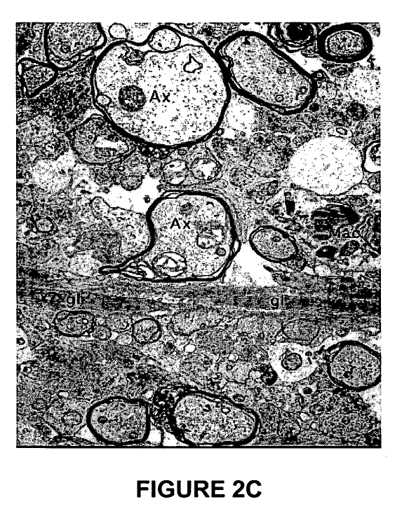

### DETAILED DESCRIPTION OF THE INVENTION

The present invention is based upon observations in marmosets and in humans that autoimmune diseases of the central nervous system occur as a result of the inability of the immune system to suppress and control viral replication. Based upon the observations disclosed herein, the present invention provides non-human animal model systems for autoimmune demyelinating diseases, such as multiple sclerosis (MS), which animal model systems will find use in the identification and characterization of therapeutic treatment modalities of neurodegenerative diseases. Within other related embodiments of the present invention are provided methodologies for the detection of markers correlative of autoimmune demyelination in humans. Each of the various embodiments of the present invention is described in detail herein below and is best understood in conjunction with the references cited herein, whether infra or supra, which are all hereby incorporated by reference in their entirety as if it were individually incorporated by reference.

### A Marmoset Animal Model System of Inflammatory and Neurodegenerative Conditions of the Central Nervous System

Within a first embodiment is disclosed a non-human experimental animal model system useful for characterizing the causal and time-dependent relationships between HHV6 infection and the occurrence of CNS inflammatory or neurodegenerative conditions. Such non-human animal model systems are exemplified by a primate animal model systems that mimic human multiple sclerosis (MS) and diabetes in a controlled fashion.

As indicated above, the present invention is based, in part, on the observation that the common marmoset (Callithrix jacchus), a New World, non-human primate, develops spontaneous autoimmunity, is susceptible to infection with human herpes virus 6 (HHV6), and is exquisitely sensitive to immunization with myelin antigens, which develops into an MS-like form of experimental allergic encephalomyelitis (EAE) that may be suitably employed for the identification and characterization of MS therapeutics and treatment regimens.

Thus, within certain embodiments are provided non-human animal model systems for inflammatory and/or neurodegenerative conditions of the central nervous system, exemplified but not limited to MS, wherein C. jacchus marmosets are infected with a herpes virus, such as, for example, one or more HHV6 variant(s) such as HHV6-A and/or HHV6-B. As described within the presently disclosed examples, infection with HHV6 is monophasic and rapidly lethal to cells in vitro yet a CNS demyelinating disorder follows in vivo infection of naïve adult marmosets with HHV6-A. Without wishing to be limited to any particular mode of action, it is believed that this and related CNS diseases appear to be associated with apoptotic cell death followed by T cell reactivity to myelin antigens, which appears subsequent to clinical disease. Apoptosis may involve glial cells (oligos, astrocytes), and also neurons as demonstrated by in vitro experiments.

Non-human animal model systems according to the present invention are correlative of autoimmune neurodegenerative diseases in humans and, thus: (1) provide an opportunity to identify the factors controlling the pathogenesis of CNS autoimmunity following exposure to HHV6-A and (2) provide a suitable system for identifying and characterizing potentially efficacious therapeutic agents for the treatment of autoimmune diseases of the central nervous system.

The presently disclosed finding that C. jacchus marmosets develop inflammatory demyelination following exposure to herpes viruses, such as variants of HHV6, provides a unique opportunity for understanding viral pathogenesis of CNS demyelination in a primate species that ubiquitously expresses functional HHV6 cellular receptors (i.e. CD46) and that has close phylogeny to man. The HHV6 infected C. jacchus marmoset animal model system will find use in further studies to reveal the factor(s) that control causal associations between CNS autoimmune demyelination in an outbred species that may exhibit differential susceptibility and a natural form of exposure (e.g., hematogenous) to HHV6, an ubiquitous virus that is not considered pathogenic in the vast majority of adult human populations.

Because C. jacchus marmosets have a natural bone marrow chimerism that allows adoptive transfer with lymphocytes, limited polymorphisms of the major histocompatibility complex (MHC) class II, and a large deletion in the MHC class I region that is a basis for their high degree of susceptibility to viral infections, especially herpes viruses, these animals may be suitably employed in the animal model systems of the present invention.

Non-human animal model systems presented herein will find utility in the identification and validation of biomarkers suitable for diagnosis of the underlying infectious causes of diseases, such as MS, that are associated with neurodegeneration, autoimmune demyelination, and diabetes. Such animal model systems may, for example, be suitably employed for such diseases in humans and are predictive of disease risk in young adults including at a pre-clinical stage.

Non-human animal models disclosed herein will find utility in modeling interactions between other ubiquitous human viruses, exposure to multiple agents and whole organisms that result in autoimmunity or states of immuno-deficiency, not only in the case of MS but also other diseases. With that regard, data obtained from, for example, the marmoset animal model may enhance the ability to model these interactions by the means of bio-informatics. Krueger et al., (2004). Non-human animal models disclosed herein will also find utility in the identification of therapeutic targets and agents for curative and preventative intervention of diseases, such as MS, that are associated with neurodegeneration, autoimmune demyelination, and diabetes that are driven by HHV6 infection.

### A Common Marmoset Animal Model System for Experimental Allergic Encephalomyelitis (EAE)

Common marmosets (white ear-tuffed marmoset, Callithrix jacchus jacchus) are New World non human primates that have been used as animal models of Parkinsonism and aging due to their ease of breeding in captivity and small size (˜400 gm at adult age). Abbott et al., Comp Med 53:339-50 (2003); Mansfield, Comp Med 53:383-92 (2003); Zuhlke et al., Toxicol Pathol 31 Suppl:123-7 (2003); Brack et al., Vet Pathol 18:45-54 (1981) and Gore et al., J Med Primatol 30:179-84 (2001). Marmosets are closely related to other primates including tamarins and humans, which all share the differential susceptibility to a number of autoimmune diseases, and spontaneous development of colitis, thyroiditis, and a wasting syndrome with kidney failure of unclear pathophysiology. Levy et al., J Comp Pathol 82:99-103 (1972) and Clapp et al., in Carcinoma of the Large Bowel and Its Precursors: Progress In Clinical and Biological Research 247-61 (Ingalls et al. eds., 1985).

Marmosets have a polymorphic MHC class II organization but a very restricted class I due to a large evolutionary deletion (Watkins et al., Journal of Immunology 144:3726-3735 (1990); Antunes et al., Proceedings of the National Academy of Sciences of the United States of America 95:11745-11750 (1998) and Cadavid et al., J. Immunol. 157:2403-2409 (1996)), which likely explains their high degree of susceptibility to a number of viruses. In addition, their phylogeny is close to that of humans and a number of immune and nervous system genes are highly conserved. Uccelli et al., J. Immunol. 158:1201-1207 (1997); Uccelli et al., Eur. J. Immunol. 31:474-479 (2001); von Büdingen et al., Immunogenetics 53:557-563 (2001); von Büdingen et al., Proc. Natl. Acad. Sci. USA 99:8207-8212 (2002); and Mesleh et al., Neurobiol Dis 9:160-72 (2002).

C. jacchus marmosets have been the subject of intense investigations of EAE in the last decade, due to their propensity to develop CNS inflammatory demyelinating disease that recapitulate the hallmark of MS clinical features and pathology. In this species, active immunization with whole human white matter, or myelin/oligodendrocyte glycoprotein (MOG) in adjuvant produce chronic, relapsing/remitting disorders of mild to moderate clinical severity which are reminiscent of typical forms of human MS. The neuropathology of acute C. jacchus EAE consists of large concentric areas of primary demyelination, macrophage infiltration, astrogliosis, and death of oligodendrocytes. Massacesi et al., Ann. Neurol. 37:519-530 (1995); Genain et al., Immunol. Reviews 183:159-172 (2001); and Brok et al., Immunol Rev 183:173-85 (2001).

Ultrastructural features of myelin breakdown are similar in marmoset EAE and human MS, suggesting common mechanisms of myelin destruction (Raine et al., Ann. Neurol. 46:144-160 (1999) and Genain et al., Immunol. Reviews 183:159-172 (2001)). The causal mechanisms underlying the marmoset, MS-like EAE lesion have been elucidated and are a complex interplay of myelin-directed autoaggressive response and pathogenic autoantibody responses. Id.

### A Marmoset Model of Diseases Associated with HHV6 Infection

Common marmosets are susceptible to infection by herpesviruses. Provost et al., J Virol 61:2951-5 (1987); Jenson et al., J Gen Virol 83:1621-33 (2002); Ramer et al., Comp Med 50:59-68 (2000); Farrell et al., J Gen Virol 78 (Pt 6):1417-24 (1997); Cox et al., J Gen Virol 77 (Pt 6):1173-80 (1996); Wedderburn et al., J Infect Dis 150:878-82 (1984); Johnson et al., Proc Natl Acad Sci USA 78:6391-5 (1981); de-The et al., Intervirology 14:284-91 (1980); Ablashi et al., Biomedicine 29:7-10 (1978); and Falk et al., Int J Cancer 17:785-8 (1976). Marmosets express a CD46 molecule that is highly homologous to human CD46 and is a target for herpesvirus infection as exemplified by infection by various strains of HHV6 including, but not limited to, HHV6-A and HHV6-B.

Using trans-well co-cultures with HHV6-infected human T cell lines, it was demonstrated as part of the present invention that marmoset lymphocytes (PBMC) can be infected in vitro with both HHV6 variants A and B. Methodologies for in vitro co-culture and infection are well known in the art and may be facilitated by stimulation of PBMC with phytohemagglutinin. Under such exemplary conditions, infection occurs within 5 to 10 days after exposure to infected human immortalized T cell lines for HHV6 variants A and B.

In vivo infection of marmosets may be achieved with HHV6 variants using various protocols as detailed herein below and summarized in Table 3 and is exemplified by the following: (1) Intravenous (i.v.) administration of the animal's own PBMC infected in vitro with HHV6-A and/or HHV6-B (as verified by such well-known techniques as immunofluorescence (IFA) and polymerase chain reaction (PCR)), followed by intravenous injection of a cell lysate containing live HHV6-A and/or HHV6-B virus variant 6-7 weeks later (see infection protocol disclosed herein for animal #190-94 and U031-00); (2) two i.v. injections of lysates from HHV6-B infected cells (such as, for example, MOLT3 cells) at 5 week intervals; (3) inoculation of cells (such as, for example, HSB2 cells) infected with HHV6-A to generate HHV6-A+ cells (e.g., HHV6-A+ HSB2 cells), followed ˜3 months later by injection of HHV6-A-infected cells (e.g., HHV6-A+ HSB2 cells; see infection protocol disclosed herein for animal #550-99) or uninfected HSB2 cells ˜3 months later (see infection protocol disclosed herein for animal #367-94). It will be understood that the selection of cells is exemplified herein by the use of MOLT3 cells or HSB2 cells but may, alternatively, include a wide range of CD46+ hematopoietic lines including, without limitation, K562, HL-60, U937, KG-1, Jurkat, MOLT4, and SupT1 cells each of which is readily available from the American Type Culture Collection (ATCC; Manassas, Va.).

C. jacchus marmosets are naïve to HHV6-A and HHV6-B, and can reliably be infected by these viruses. Repeated infection of adult animals with HHV6-A produces a mild, chronic relapsing CNS disease with pathologically, perivascular inflammatory demyelination similar to MS. Thus, the animal model system presented herein provides a causal link between a ubiquitous human virus to a chronic disorder mimicking MS, and affords a model for characterizing interactions between such microbes and complex neuro-immune responses in outbred species.

HHV6 infection by both variants A and B, which are capable of persistence and replication in marmosets as in humans, may cause transient immunosuppression. Only HHV6-A infestation, however, is believed to result in MS-like CNS inflammatory demyelination. Without limitation to any specific mechanistic theory, potential explanations include preferred CNS tropism for this variant and/or lytic or apoptotic effects on glial cells. Mimicry with myelin antigens does not appear to be a primary or causal mechanism for inflammatory CNS damage in this animal model system, although delayed T cell auto-reactivity may play a role in perpetration of chronic disease.

The demonstration of the present invention that CNS demyelination develops de novo after certain timed exposures to HHV6 in individuals of an outbred primate species is critical for research into temporal and mechanistic relationships between HHV6 infection and diseases of the central nervous system associated with neurodegeneration and demyelination, such as multiple sclerosis, owing to the presently disclosed protocols of infection that closely approximate the human condition.

Depending upon the route of administration selected, initial infection may be asymptomatic or nearly asymptomatic. Typically, however, re-exposure of animals to a second inoculation of live HHV6 virus, such as HHV6-A virus, rapidly leads to the development of weight loss and hypotonic paralysis with sensory deficits. See, for example, data presented herein for animals 190-94 and U076-03.

Neuropathology and/or analysis of cerebral spinal fluid (CSF) commonly evidences breakdown of the central nervous system (CNS) blood brain barrier and inflammation in animals receiving repeated inoculations of replicating HHV6-A. Demyelination indistinguishable from that seen in marmoset experimental allergic encephalomyelitis (EAE) may be evident after the second inoculation (see, e.g., animal #190-94), which animal also presented with a corresponding MRI-visible (magnetic resonance imaging), T2-weighted hyper-intense brain stem lesion reminiscent of previously described pathology associated with viral CNS infections (Raine et al., J Neuropathol Exp Neurol 32:19-33 (1973); Raine, in Textbook of Neuropathology 627-714 (Davis et al. eds., 1997); and Matsumoto et al., Acta Neurochir 141:439-40 (1999)). The presence of HHV6 virus may be demonstrated by immunohistochemistry in the vicinity of inflammatory infiltrates. In contrast, HHV6-A is not typically detected by either PCR or immunohistochemistry in histologically normal CNS tissue, spleen, lymph nodes, or other peripheral tissues. Cells of the CNS that become infected with HHV6 virus may further undergo a process of programmed cell death (i.e. apoptosis).

### Methods for Monitoring HHV6-Induced Inflammation and Inflammatory CNS Demyelination

Within further aspects of the present invention are provided methods for monitoring immune responses, including T cell and antibody responses, to viral antigens in the marmoset animal model system. Using, for example, standard proliferation assays with viral extracts as antigen, T cell reactivity (e.g., reactivity in PBMC or lymphoid organs) may be detected.

The present invention further provides flow cytometric methods for the detection viral infection based upon the detection of virus-specific immunoglobulin responses, in particular IgG and IgM responses. This aspect of the present invention will find utility in the detection of a wide range of viral infections, in particular those viral infections that elicit a humoral immune response. Thus, for example, the flow cytometric methods disclosed herein will be useful in the detection of viral infections wherein the viral agent is selected from the group consisting of HHV6, HHV7, HHV8, CMV, EBV, HSV, JC, BK, and SV40. Other viral infections may also be detected by the methods disclosed herein.

The flow cytometric methods presented herein are a substantial improvement over existing ELISA- and PCR-based methodologies available in the art and are highly specific for the particular viral agent to be detected. These methods are based upon the observation that anti-viral antibodies directed against and that specifically bind to viral antigens that adopt unique, non-native post translational modifications and conformations on the surface of infected cells. Such unique viral antigen species remain undetected by ELISA and PCR techniques.

Within a specific embodiment, it is disclosed that IgG antibody reactivity may, for example, be assessed by flow cytometry of serum on cell lines infected with HHV6-A and/or HHV6-B, respectively, using serum dilutions of 1:50-1:100, and a fluorescently labeled (e.g., fluorescein (FITC) or phycoerythrin (PE)) anti-monkey IgG secondary antibody. Such methodology typically detects antibody reactivity after the first viral exposure with increased antibody titers after a second, or subsequent, inoculation. Antibody (IgG) reactivity in animals is typically specific to the infecting viral variant, and is not reactive against other HHV6 variant(s) or against un-infected cell lines.

The presence of HHV6 DNA can also be monitored serially by nested PCR reactions using oligonucleotides directed against various elements of the viral genome. Consistent with the known tropism of HHV6 variants, HHV6-B but not HHV6-A may be detected in the blood of infected animals. In contrast to blood (HHV6-B) and CNS(HHV6-A detected by IHC), viral persistence or replication is typically not detected in other organs.

Viral infections can result in molecular mimicry, a phenomenon by which the host's immune system recognizes a viral peptide that resembles a myelin protein peptide thereby triggering an immune attack. See, for example, Fujinami et al., Science 230:1043-1045 (1985) and Oldstone, Faseb Journal 12:1255-1265 (1998). Such homology to an immuno-dominant peptide of myelin basic protein (MBP) was recently described within the HHV6 U24 protein. Tejada-Simon et al., Ann Neurol 53:189-97 (2003) and Cirone et al., J Med Virol 68:268-72 (2002). It is believed that molecular mimicry may lead to cross-activation of MBP-reactive T cell clones, as demonstrated for other viruses, and may underscore a possible mechanism for triggering MS attacks, or perpetrating disease. Wucherpfennig et al., Cell 80:695-705 (1995); Talbot et al., Curr Top Microbiol Immunol 253:247-71 (2001) and Lang et al., Nat Immunol 3:940-3 (2002).

As part of the present invention, it is now disclosed that T cell mimicry may occur in HHV6-inoculated animals. Animals may, for example, exhibit reactivity to Myelin oligodendrocyte glycoprotein (MOG), myelin basic protein (MBP), other HHV6 antigens, and/or peptides thereof. Serial blood samples may be obtained from animals and peripheral T cell immune reactivity (PBMC) to lectins (PHA) monitored. Typically, a second HHV6 inoculation may be followed by a transient state of immunosuppression (evidenced by decreased reactivity to PHA), and later by appearance of reactivity to a viral antigen such as MOG and/or MBP.

Apoptosis or death of oligodendrocytes and neurons has been suggested to participate in the pathogenesis of the lesions MS and EAE. Raine, J Neuroimmunol 77:135-52 (1997b); and Lucchinetti et al., Ann. Neurol. 47:707-717 (2000). HHV6 variants may also be toxic to glial cells such as astrocytes in CNS, although potential protective effects have been also reported. Kong et al., J Neurovirol 9:539-50 (2003); De Bolle et al., Clin Microbiol Rev 18:217-45 (2005); and Donati et al., J Virol 79:9439-48 (2005). Animals, such as marmosets, infected with HHV6 and characterized by inflammatory infiltrates may be further analyzed by the TUNEL reaction and/or staining for caspase 3 on sections of brain from HHV6-infected animals. These assay systems are useful in demonstrating the presence of apoptotic cells, such as glial and/or neuronal cells, in the vicinity of lesions.

Apoptosis or programmed cell death is marked by a series of characteristics including loss of cell volume, zeiosis, clumping of chromatin and nuclear fragmentation into apoptotic bodies. There are several methods known in the art that can be used to quantitate apoptosis by flow cytometry. One of the most common methods is to use propidium iodide to stain the DNA and look for the sub-diploid population of cells from a cell cycle profile. The most commonly used dye for DNA content/cell cycle analysis is propidium iodide (PI). PI intercalates into the major groove of double-stranded DNA and produces a highly fluorescent adduct that can be excited at 488 nm with a broad emission centered around 600 nm. Since PI can also bind to double-stranded RNA, cells are typically treated with RNase for optimal DNA resolution. Other well known flow cytometric based methods include the TUNEL assay, which measures DNA strand breaks and Annexin V binding, which detects relocation of membrane phosphatidyl serine from the intracellular surface to the extracellular surface. In addition, activity of the cysteine protease, caspase (typically caspase-3), may be assayed as a measure of apoptosis. Caspase can be detected using a fluorogenic substrate (Pharmingen). Microscopic examination and detection of DNA laddering by gel electrophoresis may be used to confirm the flow cytometric results.

Assay systems have also been described, and are well known in the art, for identifying cells and cell populations undergoing the process of necrosis. See, for example, Dive et al., “Analysis and Discrimination of Necrosis and Apoptosis (Programmed Cell Death) by Multiparameter Flow Cytometry,” Bioch Biophysica Acta 1133:275-285 (1992).

As part of the present invention, it was observed that apoptosis and necrosis are induced by HHV6-A in oligodentrocytes (TC620) as well as CRT (astrocytes) and neurons (SK-N-SH). These data are summarized in Table 1.

TABLE 1
Apoptosis and Necrosis on Day 3 in HHV6-A Infected Cell-lines

| Cell Line | Sample No. | Cell Death | HSB Only | HSB-HHV6-A |
|---|---|---|---|---|
| SK-N-SH | 1 | Necrosis | 3.12 | 22.02 |
| (Human Neurons) | | Apoptosis | 1.98 | 10.85 |
| | 2 | Necrosis | 3.9 | 38.76 |
| | | Apoptosis | 3.24 | 9.83 |
| | 3 | Necrosis | 4.31 | 32.3 |
| | | Apoptosis | 4.93 | 13.72 |
| CRT (Astrocytes) | 1 | Necrosis | 2.53 | 14.17 |
| | | Apoptosis | 4.88 | 23.68 |
| | 2 | Necrosis | 3.29 | 11.16 |
| | | Apoptosis | 8.05 | 26.19 |
| | 3 | Necrosis | 5.2 | No data |
| | | Apoptosis | 4.83 | No data |
| 3T3 (Mouse | 1 | Necrosis | no data | 3.19 |
| Fibroblast. Lacks | | Apoptosis | no data | 2.9 |
| CD46 expression | 2 | Necrosis | 1.57 | 5.16 |
| | | Apoptosis | 1.12 | 3.21 |
| | 3 | Necrosis | 1.43 | 3.3 |
| | | Apoptosis | 1.04 | 2.46 |

Thus, the present invention futher provides methods for the detection of HHV6-A mediated cell death, including programmed cell death (apoptosis), necrosis, cytokine-mediated cell death, cell lysis and toxicity in a patient sample such as blood, cerebral spinal fluid, and/or urine. Methods according to this embodiment comprise the step of assessing cell death, as applied to oligodentrocytes, astrocytes, and neurons as discussed above, as well as a wide range of cells exemplified herein. These methods can be applied to a wide range of tissue samples and cell types and will find utility in the detection of a wide variety of virally-induced disease states as presented in Table 2.

TABLE 2
Autoimmune Diseases and Corresponding Tissue/Cell
Types Affected that may be Assayed for Disease Etiology
by Virtue of HHV6-A Mediated Cell Death

| Autoimmune Disease | Tissue/Cell Type |
|---|---|
| Diabetes | Islet Cells |
| Lupus | Kidney Cells |
| Vasculitis | Endothelial Cells |
| Rheumatoid Arthritis | Synovial Cells |
| Dermititis | Epithelial Cells |
| Myositis | Myocytes, Myoblasts |
| Thyroiditis | Endocrine Cells |
| Addison's | Adrenal Cells |
| Anemia | Erythrocytes |
| Immune Deficiency Syndromes | T Cells, B Cells, NK Cells |
| Thrombocytopenia | Megakaryocytes |
| Hepatitis | Hepatocytes |
| Inflammatory Bowel Disease | Epithelial Cells of the |
| (Crohn's, Colitis) | Intestine and Stomach |
| Parkinson's Disease | Basal Ganglia and Substantia |
| | Nigra Neurons |
| Alzheimer's Disease | Temporal Lobe and Hippocampal |
| | Neurons |
| ALS | Spinal Cord Motor Neurons |
| Sensory Neuropathy | Dorsal Ganglia Neurons |

### A Non-Human Animal HHV6-B Infected Model System for Diabetes

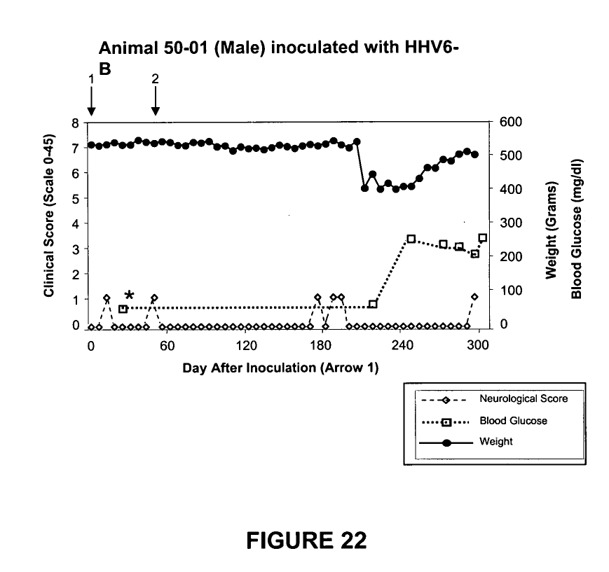

Within another embodiment of the present invention is provided a non-human animal model system for diabetes. This aspect of the present invention is based upon the observation that the HHV6-B herpesvirus variant is capable of inducing weight loss and elevated blood sugar values in an infected marmoset (see FIG. 22; animal #50-01) following a series of two inoculations with this virus. Animal model systems disclosed herein exhibit a substantial rise in urinary and/or blood sugar content and experience an abrupt weight loss.

Thus, provided herein are marmoset animal model systems of diabetes generated by the infection of a marmoset with a herpesvirus variant selected from the group consisting of HHV6-A and HHV6-B wherein the animal model is characterized by a urinary and/or blood sugar content of about between about 100 mg/dl and about 5,000 mg/dl, more typically between about 100 mg/dl and about 1,000 mg/dl, still more typically between about 150 mg/dl and about 500 mg/dl and a weight loss at day 210 of between about 15-50%, more typically between about 20 and 30%. Specifically exemplified herein is a marmoset animal model system of diabetes wherein the animal was exposed to HHV6-B, exhibited a urinary sugar content of about 1,000 mg/dl and a weigh loss of ˜27% initial weight at around 210 days after initial inoculation with HHV6-B. In contrast to animals inoculated with the HHV6-A herpsesvirus variant, animals infected with HHV6-B do not develop a substantial neurological deficit or central nervous system pathology.

It is further contemplated that the present non-human animal model systems may be suitably extended to a wide variety of viruses that may be associated with the onset of diabetes including, but not limited to, one or more coronavirus, rheovirus, adenovirus, paramyxovirus, and/or coksackie virus.

### Transgenic Animal Model Systems for Multiple Sclerosis and Other Autoimmune Diseases

Within other embodiments of the present invention are provided non-human transgenic animal model systems for multiple sclerosis and other related autoimmune diseases of the central nervous system that are characterized by demyelination. A wide variety of animal species are contemplated in connection with these embodiments of the present invention. For example, provided herein are non-human transgenic mouse, zebrafish, drosophila, and nematode animal model systems, wherein the animal comprises a transgene encoding CD46 and is infected with and/or exposed to a herpesvirus.

Transgenic mouse animal model systems according to the present invention may be generated by reference to methodologies that are readily available in the art. See, for example, the methodologies described in Hogan et al., “Manipulation the mouse embryo: A laboratory Manual” (Cold Spring Harbor Laboratory, Cold Spring Harbor, N.Y., 1986); Palmiter et al., Nature 300:611-15 (1982); Ebert et al., Mol. Endocrin. 2:277-83 (1988); Sutrave et al., Gene Dev. 4:1462-72 (1990); Pursel et al., Theriogenology 45:348 (1996); U.S. Pat. Nos. 6,323,390, 6,218,597, 6,137,029, 6,156,727, 6,127,598, 6,111,166, 6,107,541, and 6,077,990, each of which is incorporated herein by reference in its entirety.

Transgenic mice comprising and expressing a human CD46 transgene have been described for the study of measles virus infection. Rall et al., Proc. Natl. Acad. Sci. U.S.A. 94:4659-4663 (1997) describe a transgenic mouse model for measles virus infection of the brain wherein the CD46 gene is transcriptionally regulated by a neuron-specific promoter. Expression of CD46 rendered primary neurons permissive to infection with MV-Edmonston. Horvat et al. (J. Virol., 70(10): 6673-6681 (1996)) describe transgenic mice that ubiquitously express human CD46. These authors placed the C-CYT2 isoform of CD45 (containing exons 1 to 6, 9 to 12, and 14) under the control of the gene promoter of the ubiquitously expressed hydroxymethyl-glutaryl coenzyme A reductase (HMGCR). This construct was microinjected into the pronuclei of B6DBA mouse ovocytes and transgenic mice were generated by standard methods. Evlashev et al. (J. Virol. 74:1373-1382 (2000) and J. Gen. Virol. 82:2125-2129 (2001)) describe a transgenic mouse model of MV-induced pathology wherein several lines of transgenic mice were generated that ubiquitously expressed in the brain the human CD46 with either a Cyt1 or Cyt2. cytoplasmic tail. Hahm et al. (J. Virol. 77:3505-3515 (2003)) describe transgenic mice that express the human signaling lymphocytic activation molecule (hSLAM) molecule under the control of the lck promoter. hSLAM was expressed on CD4+ and CD8+ T cells in the blood and spleen and on CD4+, CD8+, CD4+ CD8+, and CD4− CD8− thymocytes. Each of these references is incorporated herein by reference in its entirety.

Exemplified herein is a transgenic mouse animal model system wherein the transgenic mouse comprises a transgene encoding CD46, wherein the transgenic mouse is infected with a herpesvirus, and wherein the herpesvirus is selected from the group consisting of HHV6-A and HHV6-B. Within certain aspects of these embodiments, the transgene encoding CD46 is ubiquitously expressed in vivo. Within alternative aspects, the transgene encoding CD46 is expressed in vivo in a tissue selected from the group consisting of brain, spinal cord, and peripheral nerve. Such transgenic mouse animal models will find use in mechanistic studies of multiple scleroses and will be employed to further characterize candidate therapeutics, including therapeutics for the treatment of multiple sclerosis, and treatment regimens, such as those identified via the marmoset MS model system described in detail herein above.

Transgenic mouse animal model systems presented herein may be achieved by a single exposure of the CD46 transgenic mouse to a herpesvirus wherein such viral exposure triggers and/or increases the severity of a central nervous system inflammatory disease. Within alternative aspects, more than one exposure of the transgenic mouse to a herpesvirus is required to trigger and/or increases the severity of a central nervous system inflammatory disease. Within yet further aspects of the present invention, the CD46 transgenic mouse is exposed to a combination of two or more viruses such as, for example (a) HHV6-A and HHV6-B; (b) HHV6 and CMV; (c) HHV6 and EBV; (d) HHV6 and VZV; (e) HHV6 and HHV8; (f) HHV6 and HIV; and (g) HHV6 and HTLV.

Viral infection may be achieved essentially as described herein for the marmoset animal model systems of herpesvirus infection. For example, an appropriate tissue sample may be withdrawn from the animal, subjected to conventional tissue culture techniques, exposed ex vivo to one or more herpesvirus variant and/or combination of viruses as indicated above, and the infected cells reintroduced into the animal. Suitable cells for such an autologous technique that may be infected include those cells that express cell-surface CD46 such as, for example, PBMC, splenocytes, and lymph node cells. Other cell-types may also be employed for herpesvirus infection. The extent of ex vivo viral infection may be monitored and assessed with a dose-response curve based on a plaque forming assay or counting viral particles in an isolate.

Typically, cells are reintroduced into the animal via intravenous injection, intra-peritoneal injection, or subcutaneous injection. Other routes of administration may be appropriate and will be determined by the artisan in view of the particular cell-type and application contemplated. As noted herein, viral infection of the animal may be successfully achieved by a single ex vivo viral exposure and reintroduction or may require one or more subsequent round(s) of ex vivo viral exposure and reintroduction, typically at intervals of about 3 to about 8 weeks. Exemplified herein are a number of infection regimens that may be suitably employed.

Transgenic mouse animal model systems disclosed herein are suitably employed for studying the potential of a candidate compound for reducing the severity of a disease of the central or peripheral nervous system such as, for example, a nervous system inflammatory disease. Typically, exposure of a CD46 transgenic mouse with one or more herpesvirus, as described herein, triggers and/or increases the severity of an inflammatory disease and/or autoimmune disorder selected from the group consisting of multiple sclerosis, diabetes, arthritis, anemia, lupus, pemphigus, thyroiditis, glomerular or interstitial nephritis, cardiomyopathy, myositis, dermatomyositis, hepatitis, and ulcerative colitis. Such herpesvirus infected CD46 transgenic animal model systems are suitable for the identification of factors mediating the direct toxicity of the herpesvirus towards a cell type such as, for example, a cell type selected from the group consisting of an oligodendrocyte, an astrocyte, and a brain cell. Toxicity may be assessed by methodology that are well know in the art and as described herein such as, without limitation, histological examination, assessment of apoptosis and/or necrosis, measurement of cytokines and other factors, and/or T cell and antibody reactivity in peripheral blood/lymphoid organs. Exemplary factors include, without limitation, cells of the immune system such as CD4+ T-cells and CD8+ T-cells.

### Transgenic Zebrafish Model Systems for Multiple Sclerosis and Other Autoimmune Diseases

Transgenic zebrafish expressing human CD46 may be produced by introducing a transgenic construct into cells of a zebrafish, typically embryonic cells or into a single embryo as described by Meng et al. Methods Cell Biol. 60:133-48 (1999) and in U.S. Patent Application Publication No. 2005/0120392, each of which reference is incorporated herein in its entirety. Transgenic constructs may, for example, be generated by modifying commercially available plasmid systems such as pDsRed2-1 (Clontech) and p-αEGFPITR as described in U.S. Patent Application Publication No. 2004/0117866 and Chou et al., Transgenic Research 10:303-315 (2001), to express human CD46. Such constructs may be integrated into the genome of a zebrafish or may be constructed as an artificial chromosome. Transgenic constructs may be introduced into embryonic cells using techniques that are known in the art such as, for example, microinjection, electroporation, liposomal delivery and particle gun bombardment. Embryos may be microinjected at the one or two cell stage or the construct may be incorporated into embryonic stem cells that can later be incorporated into a growing embryo.

Embryos or embryonic cells may be obtained as described in Rubenstein et al., U.S. Patent Application Publication Nos. 2005/0120392, 2002/0187921 and 2004/0143865 and in Tsai U.S. Patent Application Publication No. 2004/0117866. Zebrafish containing a CD46 transgene may be identified by numerous methods such as probing the genome of the zebrafish for the presence of the transgene construct by Northern or Southern blotting. Polymerase chain reaction techniques may also be employed to detect the presence of the transgene.

Expression of a reporter protein may also be detected by methods known in the art. For example, RNA can be detected using any of numerous nucleic acid detection techniques. Alternatively, an antibody can be used to detect the expression product or one skilled in the art can visualize and quantify expression of a fluorescent reporter protein such as GFP. As used herein, a reporter protein is any protein that can be specifically detected when expressed. Reporter proteins are useful for detecting or quantifying expression from expression sequences. For example, operatively linking nucleotide sequences encoding a reporter protein to a tissue specific expression sequence allows one to study lineage development. In such studies, the reporter protein serves as a marker for monitoring developmental processes.

Many reporter proteins are known to those of skill in the art. These include, but are not limited to, β-galactosidase, luciferase, and alkaline phosphatase that produce specific detectable products. Fluorescent reporter proteins can also be used, such as green fluorescent protein (GFP), enhanced green fluorescent protein (eGFP), reef coral fluorescent protein (RCFP), cyan fluorescent protein (CFP), red fluorescent protein (RFP) and yellow fluorescent protein (YFP). For example, by utilizing GFP or RCFP, fluorescence is observed upon exposure to ultraviolet, mercury, xenon, argon or krypton arc light without the addition of a substrate.

The use of reporter proteins that, like GFP, are directly detectable without requiring the addition of exogenous factors may be preferred for detecting or assessing gene expression during zebrafish development. A CD46 transgenic zebrafish embryo, carrying a construct encoding a reporter protein and a tissue-specific expression sequence, provides a rapid, real time in vivo system for analyzing spatial and temporal expression patterns.

### CD46-Based Compositions

Other embodiments of the present invention provide compositions comprising CD46 variant selected from the group consisting of (a) a soluble CD46, (b) a cell associated CD46, and (c) an artificial delivery system associated CD46; wherein the composition is effective in reducing the severity of a disease selected from the group consisting of multiple sclerosis and/or other autoimmune and immune-mediated inflammatory diseases of the brain or other target organs; wherein the soluble CD46 is produced in recombinant form, as a full-length polypeptide or as a truncated variant; and wherein the artificial delivery system is either a liposome or a vesicle. Within certain aspects, such compositions are effective in the treatment of a neurodegenerative disorder and/or a tumor.

### Methods Employing Non-Human Animal Model Systems of CNS Inflammatory and/or Neurodegenerative Conditions

The present invention further provides, in various embodiments (1) methods for detecting a patient at risk for developing a disease; (2) methods for evaluating in a patient, such as a human patient, the existence of antibodies or cellular responses that result in neutralization of herpesvirus-mediated infections, such as HHV6-mediated infections; (3) methods for identifying a compound effective in reducing the severity of herpesvirus-mediated toxicity in a cell within a patient sample; (4) methods for evaluating the therapeutic potential of candidate compounds or other interventions that antagonizes the development of detrimental autoantibodies; and (5) methods for detecting in a patient the risk of infection with a ubiquitous virus in a disease state such as multiple sclerosis and/or another autoimmune disorder. Each of these methods is described if further detail herein and within the Examples.

Within one embodiment, the present invention provides methods for detecting a patient at risk for developing a disease such as, for example, multiple sclerosis and/or other autoimmune and immune-mediated inflammatory diseases of the brain or other target organs. Within certain aspects, such methods comprise the steps of: (a) isolating from the patient a biological sample suspected of comprising an antibody that specifically binds to human CD46; (b) contacting the biological sample with human CD46 or a variant thereof (e.g., CD46 adsorbed to a solid matrix or a cell expressing CD46) for such a time and under such conditions as required to achieve a first complex comprising the antibody that specifically binds to human CD46 and the cell expressing human CD46; (c) contacting the complex with a secondary anti-human antibody, wherein the secondary antibody comprises a detectable tag, for such a time and under such conditions as required to achieve a second complex comprising the secondary anti-human antibody specifically bound to the first complex; and (d) detecting the detectable tag on the bound secondary antibody.

Typically, the detectable tag on the secondary antibody is detected by fluorescence activated cell sorting analysis or other method wherein a detection tag is used to reveal the presence of the detectable tag on the secondary antibody. Detectable tags may, for example, be fluorescent tags or radioisotopes. Within certain aspects, methods according to these embodiments may be suitably employed for identify a patient wherein an active destructive process is linked to or concomitant with herpesvirus replication, including HHV6 replication, and activity is ongoing. By such methods, early treatment regimens may be initiated in the patient whereby full development of a disease such as multiple sclerosis, chronic fatigue syndrome, and other related disorder is prevented.

Related embodiments of the present invention provide methods for evaluating in a patient, such as a human patient, the existence of antibodies or cellular responses that result in neutralization of herpesvirus-mediated infections, such as HHV6-mediated infections. Similar methods are provided that permit the evaluation of such patients for failure to produce an antibody and/or T cell response resulting in early or delayed organ-specific autoimmunity, including multiple sclerosis and diabetes. By these methods, antibodies, such as neutralizing antibodies, or cellular responses are detected and correlated with the risk of a patient developing a disease of the central nervous system, such as multiple sclerosis and/or the risk of a patient developing an autoimmune disorder selected from the group consisting of diabetes, arthritis, anemia, lupus, pemphigus, thyroiditis, glomerular or interstitial nephritis, cardiomyopathy, myositis, dermatomyositis, hepatitis, and ulcerative colitis.

Other embodiments of the present methods include methods for identifying a compound effective in reducing the severity of herpesvirus-mediated toxicity in a cell within a patient sample, wherein such methods comprise the steps of (a) administering to a non-human animal model system, as described herein, a candidate compound and (b) determining in a cell within a patient sample whether the herpesvirus-mediated toxicity is reduced in severity. Typically, such herpesvirus-mediated toxicity is correlative of a neurodegenerative disease selected from the group consisting of multiple sclerosis, Parkinson's disease, Alzheimer's disease, and cerebellar degeneration. Exemplary cells within a patient sample include neurons and cells within a patient's serum, blood, cerebral spinal fluid (CSF), and/or other patient samples. Measurements of cellular toxicity include, without limitation, a lytic effect, cytokine-mediated cell death, and apoptosis.

Also provided are methods for evaluating the therapeutic value of a compound or other intervention that antagonizes the development of detrimental autoantibodies the generation of which is induced by exposure to a herpesvirus such as, for example, HHV6-A and/or HHV6-B. Related methods are provided for evaluating the therapeutic value of a compound or other intervention that favors the development of a beneficial autoantibody. Additional methods are provided for evaluating the therapeutic value of a compound or intervention that alters the immune system via its cellular responses such that detrimental autoantibodies are antagonized or beneficial autoantibodies are agonized.

The present invention also provides, in other embodiments, methods for detecting in a patient the risk of infection with a ubiquitous virus in a disease state such as multiple sclerosis and/or another autoimmune disorder wherein the patient is susceptible to immunosuppression, transplant, AIDS, and/or other immunodeficiency.

### Markers and Methods for the Detection of Autoimmune Diseases of the Central Nervous System

As discussed in the examples below, it has been reported that patients receiving natalizumab in combination with interferon beta 1-a (IFN-β) for diseases such as relapsing remitting multiple sclerosis (MS) and Crohn's disease, have exhibited progressive multifocal leukoencephalopathy (PML), a disease the etiology of which has been associated with infection with the HIV and JC viruses. See, for example, Bossolasco et al., “Prognostic Significance of JC Virus DNA Levels in Cerebrospinal Fluid of Patients with HIV-Associated Progressive Multifocal Leukoencephalopathy” Clinical Infectious Diseases 40(5):738-744 (2005). Thus, PML is usually observed in immunosuppressed individuals (for example, AIDS, transplant patients), as are opportunistic infections with other common human pathogens. B cells may participate in the pathogenesis of PML by transporting the virus from kidney to brain. The disease is thought to be mediated through replication of JC virus in oligodendrocytes.

Based upon these observations with human patients, the present invention further provides markers and methods for assessing the immunological properties of lymphocyte subsets in patients developing PML after treatment with one or more therapeutic modality. Thus, the presently described embodiments provide markers and methods for the identification of patients, including human patients, that are susceptible to complications, such as progressive multifocal leukoencephalopathy (PML) or other encephalitis, when under treatments such as Muromonab-CD3 (Johnson & Johnson), Abciximab (Centocor), Rituximab (Biogen IDEC), Daclizumab (Protein Design Labs), Basiliximab (Novartis), Palivizumab (MedImmune), Infliximab (Centocor), Trastuzumab (Genentech), Gemtuzumab (Wyeth), Alemtuzumab (Millennium/ILEX), Ibritumomab (Biogen IDEC), Adalimumab (Abbott), Omalizumab (Genentech), Tositumomab-I131 (Corixa), Efalizumab (Genentech), Cetuximab (Imclone Systems), and Bevacizumab (Genentech) or other strong immunosuppressive biologicals currently in use for a wide range of virus-related immune disorders such as, for example, multiple sclerosis.

Thus, for example, the present invention provides flow cytometric methods for detecting the risk of infections with ubiquitous viruses in autoimmune disorders, including multiple sclerosis, diseases treated with immunosuppression, transplant, AIDS, and other conditions of immunodeficencies, other neurodegenerative or organ-specific pathologies. Such methods comprise the step of measuring, in a patient sample such as peripheral blood, the CD19 and CD3 levels and/or ratios, CD4+CD25+ populations, levels of regulatory T cells, and/or levels of CD8, and correlating those levels and/or ratios with the risk that that patient will present with virus-related and cancerogenic complications. A wide number of virus-related complications are presently contemplated such as those complications the etiology of which is associate with a virus such as, for example, JC, HHV6, EBV, VZV, HHV7, HHV8, CMV, HSV I, and HSV II.

Heparinized blood and clotted serum may be collected a patient undergoing a therapeutic regimen and stained for flow cytometry (FACS) analysis according to the manufacturer's instructions using one or more fluorescently-tagged primary antibody such as, for example: CD3-FITC/PE/PerCp: SP34, CD4-FITC/PE: L200 (BD Pharmingen), CD8-FITC: SFCI21Thy2D3 (Beckman Coulter), CD19-PE: 4G7, and CD25-FITC: 2A3 (Becton-Dickinson).

Typically, white blood cell (WBC) counts, lymphocyte counts, and absolute counts of CD3⁺CD4⁺, CD3⁺CD8⁺, and CD 19⁺ cells are unaffected in virus-related complications measured by the present methods. In contrast, however, a reduction in CD3⁺CD8⁺ cell counts, an increase in absolute CD19⁺ counts, an increase in the relative proportion of CD19⁺ cells (mature B cells), and an increase in CD19/CD3 rations indicates an increase the risk that a patient will exhibit virus-related and cancerogenic complications.

In addition, CD4⁺CD25⁺ T cells, which include T cells with regulatory activity (Treg) are reduced, or virtually absent, in patients developing PML as evidenced by absolute counts of CD4⁺CD25⁺ cells. It is believed that suppression of Treg populations occurs, despite relatively preserved total lymphocytes and CD4⁺ T cells, and that B cell populations in these patients tend to increase, especially in proportion of CD8⁺ (suppressor) cells. Without being limited to any particular mechanistic theory, it is believed that loss of T regulatory activity may be responsible for deficient control of B cell activity and trafficking in these patients, and inability to prevent replication of dormant and usually benign viruses such as the JC virus. The state of “functional immunodeficiency” may resulting from a loss of T regulatory activity may also affect the ability of other ubiquitous pathogens to reactivate, such as in cases of PML.

Measurement of immunological markers such as T cell subsets, particularly CD19⁺ and CD4⁺CD25⁺, will find utility for the detection of patients at risk for the development of therapeutically-induced complications. In particular, the data presented herein support the assessment and use of CD19/CD3 ratios, CD4⁺CD25⁺ populations, CD8⁺ and CD3⁺ cell counts as markers for monitoring the risks of patients with MS and other autoimmune disorders treated with therapeutic modalities such as natalizumab.

All references cited herein, whether infra or supra, are hereby incorporated by reference in their entirety.

### EXAMPLES

The following Examples are offered by way of illustration, not limitation.

### Example 1

### Infection of Marmoset Cells In Vitro

This Example demonstrates the susceptibility of marmoset lymphocytes (PBMC) to infection in vitro with HHV6 variants A and B.

Common marmosets are susceptible to infection by herpesviruses (Provost et al., J Virol 61:2951-2955 (1987); Jenson et al., J Gen Virol 83:1621-1633 (2002); Ramer et al., Comp Med 50:59-68 (2000); Farrell et al., J Gen Virol 78(6):1417-1424 (1997); Cox et al., J Gen Virol 77(6):1173-1180 (1996); Wedderburn et al., J Infect Dis 150:878-882 (1984); Johnson et al., Proc Natl Acad Sci USA 78:6391-6395 (1981); de-The et al., Intervirology 14:284-291 (1980); Ablashi et al., Biomedicine 29:7-10 (1978); Falk et al., Int J Cancer 17:785-788 (1976)), and express a CD46 molecule that is highly homologous to the human HHV6 receptor. Murakami et al., Biochem J 330:1351-1359 (1998). Using trans-well co-cultures with HHV6-infected human T cell lines, it was demonstrated that marmoset lymphocytes (PBMC) can be infected in vitro with both HHV6 variants A and B.

### Example 2

### Infection of Marmosets In Vivo

This Example demonstrates the susceptibility of C. jacchus marmosets to in vivo infection with HHV6 variants A and B.

Seven adult marmosets were infected with HHV6 in vivo using various protocols (summarized in Table 3 below): (1) intravenous inoculation of the animal's own PBMC infected ex vivo with HHV6-A or HHV6-B (as verified by IFA and PCR), followed by intravenous injection of a cell lysate containing identical live virus variant 6-8 weeks later; (2) two intravenous injections of viral lysates from MOLT3 HHV6-B-infected cultures at 5 weeks interval; and (3) one inoculation of HSB2 cells infected with HHV6-A (HHV6-A⁺ HSB2), followed by injection of either infected or uninfected cells 3 months later.

TABLE 3
Infection of Common Marmosets with HHV6 Variants

| | Age | | | | Clinical | Euthanasia | Inflam- | |
|---|---|---|---|---|---|---|---|---|
| Animal ID | (yrs) | Sex | Infusion 1 (day 0) | Infusion 2 (day) | signs | (day) | mation | Demyelination |
| 190-94 | 7 | F | 8 × 10⁶ HHV6-A⁺ · PBMC | HHV6-A⁺ HSB2 Lysate (42) | Yes | 68 | + | + |
| 125- | 9 | F | HHV6-B⁺ · MOLT3 Lysate | HHV6-B⁺ MOLT3 Lysate (35) | Minor | 188 | − | − |
| 550-99 | 3 | M | 15 × 10⁶ HHV6-A⁺ · HSB2 cells | 20 × 10⁶ HHV6-A⁺ · HSB2 cells (88) | Yes | 131 | + | − |
| 367-94 | 8 | F | 16 × 10⁶ HHV6-A⁺ · HSB2 cells | 20 × 10⁶ HHV6-A⁻ HSB2 cells (91) | No | 150 | + | − |
| U031-00 | 3 | M | HHV6-A⁺ · PBMC | HHV6-A⁺ · HSB2 Lysate (49) | Yes | 165 | +* | −** |
| U054-01 | 2 | M | HHV6-B⁺ PBMC | HHV6-B⁺ · MOLT3 Lysate (49) | No | 196 | + | − |
| U076-03 | 1 | M | 2.3 × 10⁶ HHV6-A⁺ PBMC | HHV6-A⁺ HSB2 Lysate (80) | Yes | 99 | + | + |

*Animal U031-00 was observed chronically (165 days), exhibited marked brain parenchymal atrophy.
**Animal U031-00, inflammation was assessed by cerebralspinal fluid (CSF) pleiocytosis and/or histology (sub-pial and/or perivascular infiltrates).

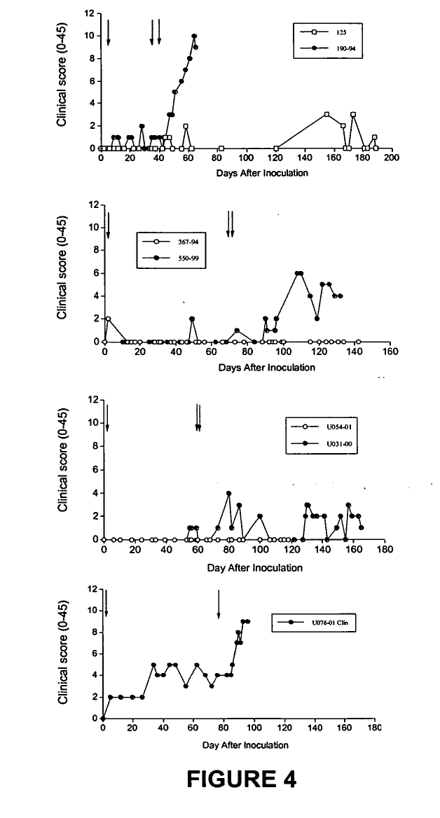

For each of these animals, initial infection was near asymptomatic. Animals re-exposed to live HHV6-A, however, rapidly developed weight loss and hypotonic paralysis with sensory deficits following the second inoculation. See, FIG. 4 Animal Nos. 190-94 and 550-99.

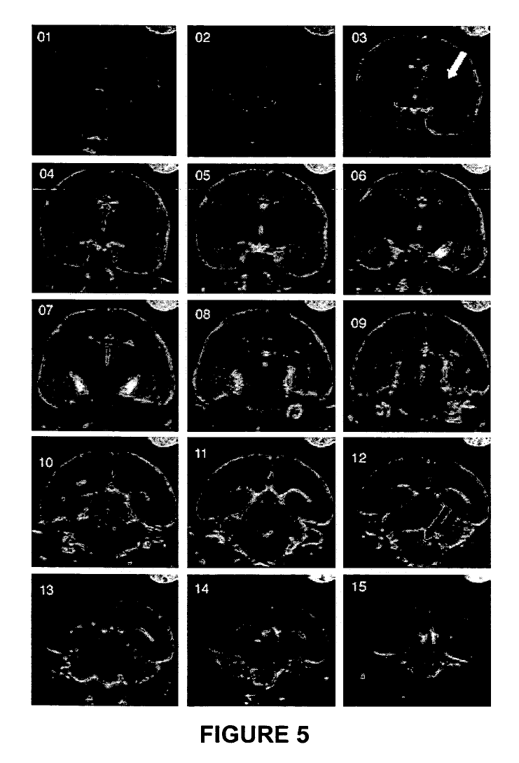

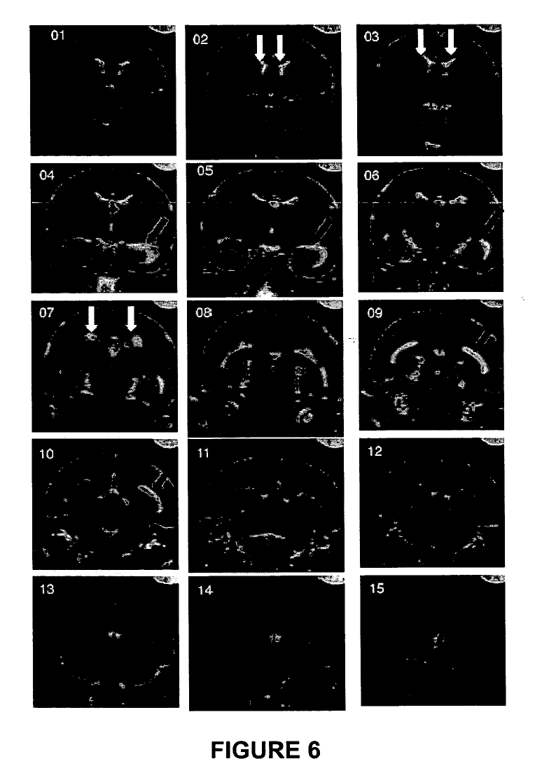

MRI imaging was performed in some of the animals at various times before and after inoculations. Positive findings included: large T2-weighted and T1-weighted hypointensities in the basal ganglia (190-94, FIG. 5); prominent regional, and to a lesser extent diffuse brain atrophy. Atrophy was evident from enlarged cerebrospinal volume including the lateral ventricles and sub-pial spaces, and was regionally predominant around hippocampal gray matter, and temporal and occipital lobes on the left side of the brain (U031-00, FIG. 6). Both these findings likely corresponded to sequellae and signatures of viral CNS infection. It is noteworthy that the marked atrophy and expansion of brain ventricular volume (FIG. 6) was observed in the animal euthanized late after the 2 viral inoculations (U031-00, 163 days), and not in the animal euthanized at an earlier time point which displayed prominent, acute demyelinating lesions but no visible atrophy (190-94, 68 days). The findings in animal U031-00 are highly reminiscent of the “ex vacuo” brain atrophy that is characteristic of MS with severe physical or cognitive impairment, and is a common outcome in the natural history of this disease regardless of initial presentation (relapsing remitting, secondary progressive and primary progressive). This MRI pattern of diffuse or regional brain atrophy in human MS is observed irrespective of the appearance of new focal MRI lesions, and, whether present early or late in the course of disease, is believed to reflect destruction and loss of periventricular white matter and/or gray matter. Brain atrophy is often considered as a paraclinical marker that signals onset of a stage of MS that follows reversible neuro-inflammation and where irreversible damage to neurons or oligodendrocytes occurs.

The above findings suggest that HHV6 infection in man could result in particular subtypes of MS that tend to involve destruction of white and/or gray matter, as opposed to more “benign” forms that are also well known to neurologists within the heterogeneous spectrum of this disease. See Pittock et al., Ann Neurol 56:303-306 (2004). Additionally (or alternatively), the involvement of basal ganglia structures in animal 190-94 and the regional atrophy in animal U031-00 suggest that the current marmoset model could also be valuable to study the causal relationships between exposure to common viruses in humans and neurodegeneration or phenotype severity in other disorders, for example chronic fatigue syndrome (CFS), narcolepsy, Parkinson's, Alzheimer's, Picks, or other forms of dementia, progressive supranuclear palsy, choreoathatosis, subacute sclerosing panencephalitis, Jacob-Creutzfeld, progressive multifocal leukiencephalopathy, late onset certain forms of focal or generalized epilepsy, Rasmussen's encephalitis, cerebellar atrophies, combined spinal cord sclerosis, MELAS and Cadasil disease.

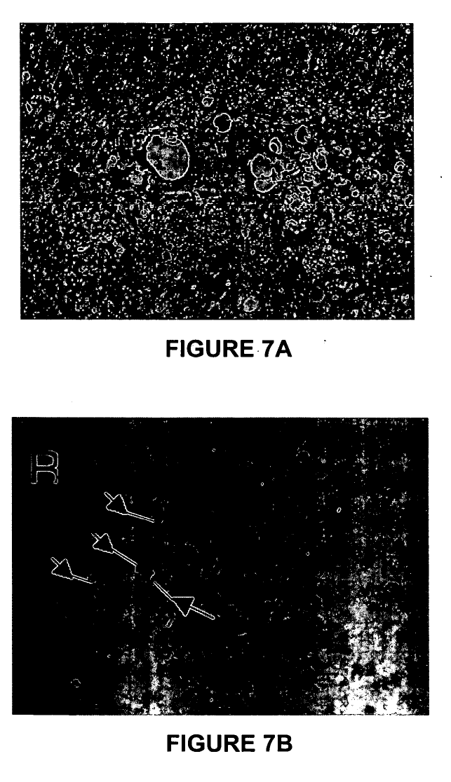

Neuropathology obtained for all animals showed perivascular or subpial infiltrates of mild inflammation in the animals that received repeated infusions of replicating HHV6-A. Inflammation was most prominent in perivascular distribution and associated with clearly visible demyelination in one animal that was sacrificed early after the second inoculation (190-94). These lesions were indistinguishable from those of marmoset EAE on routine stains, and could also be detected in vivo by MRI (FIG. 5). The presence of HHV6 virus was demonstrated by immunohistochemistry in the vicinity of inflammatory infiltrates in animal 190-94. See, FIG. 7A-7B. In contrast, HHV6 was undetectable by either PCR or immunohistochemistry in histologically normal CNS tissue, or in spleen, lymph nodes and several peripheral tissues examined. These data suggest that appearance of CNS pathology with fully developed, MS-like inflammatory demyelinating lesions may require viral persistence and/or replication. Inducement of significant clinical disease or neuropathology using HHV6-B lysates, or a single injection of HHV6-A⁺ cells was not so far detected in the animals subjected to this protocol (i.e. Nos. 125 and 367-94)

### Example 3

### Immuno-Reactivity to Viral Antigens

This Example discloses methodology for monitoring T cell and antibody responses to HHV6-Antigens.

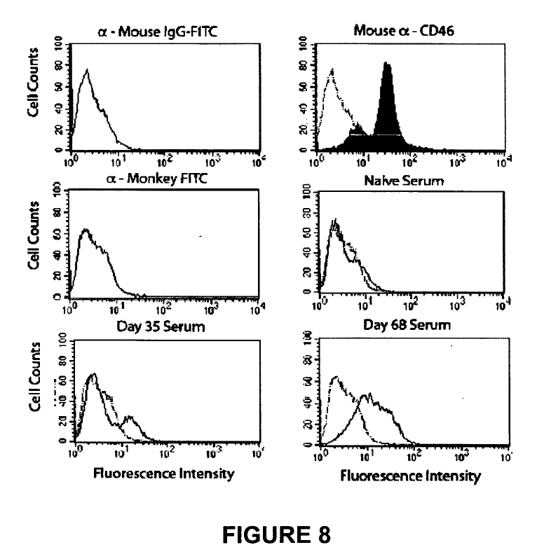

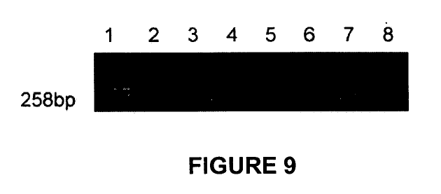

Methods were developed to serially monitor T cell and antibody responses to viral antigens. Preliminary evidence indicated that marmosets in the colony were naïve to HHV6, and that antibody reactivity appeared after inoculation. (See, FIG. 8.) No significant T cell reactivity was detected in PBMC or lymphoid organs. The presence of HHV6 DNA was monitored serially by nested PCR methodology. Consistent with the known tropism of HHV6 variants, HHV6-B, but not HHV6-A, was detected in the blood of infected animals. (See, FIG. 9.)

Importantly, while clear serum (IgG antibody) reactivity to HHV6-infected cells was observed to appear during the weeks following inoculation and was readily detected using FACS analysis, no IgG reactivity at all was detected in any of these sera by standard ELISA methods utilizing purified viral lysates coated on solid support, Strain Z-29 (Applied Biosciences, Foster City, Calif.) used according to manufacturer instruction; and data from our own laboratory using viral-extract-coated plates) (Table 4).

TABLE 4
Lack of serum IgG reactivity of HHV6-A and B-infected animals against
HHV6-At baseline and various times after infection until euthanasia,
using ELISA wells coated with purified viral extracts

| 1 | 2 | 3 | 1 | 2 | 3 |
|---|---|---|---|---|---|
| Blank | 190 (1/12/01) | 550 (7/01/02) | 0.0406 | 0.0414 | 0.0411 |
| Pos | 190 (1/12/01) | 550 (7/01/02) | 4.0000 | 0.0427 | 0.0426 |
| Pos | 190 (2/1/02) | 550 (10/23/02) | 3.1701 | 0.0488 | 0.0453 |
| Neg | 190 (2/1/02) | 550 (10/23/02) | 0.0450 | 0.0408 | 0.0403 |
| Neg | 125 (1/9/02) | 367 (7/1/02) | 0.0418 | 0.0422 | 0.0409 |
| Neg | 125 (1/9/02) | 367 (7/1/02) | 0.0419 | 0.0405 | 0.0412 |
| PP1095 | 125 (7/12/02) | 367 (10/29/02) | 3.0592 | 0.0410 | 0.0415 |
| PP1095 | 125 (7/12/02) | 367 (10/29/02) | 3.1588 | 0.0611 | 0.0409 |

Animal number (serum added as primary antibody) and date of phlebotomy. Blank, no serum; pos, positive control provided with ELISA kit; neg, negative control; PP1095, serum from patient with primary progressive (PP) MS that shows very strong HHV6 reactivity. Positive samples above background are highlighted in grey tone. (Left panel). Optical density (OD) readings from ELISA plate. Serum dilution 1:20. (Right panel).

For each of the 4 animals studied here, the sample from day of euthanasia is highlighted in bold type. Compare data from animal 190-94 (negative OD reading in ELISA for IgG) with results in FIG. 8 (positive FACS staining for IgG reactive to HHV6-infected HSB2 cells).

In addition to IgG, serum IgM reactivity was studied in all animals. IgM reactivity is considered to reflect an early phase of the primary immune response, and although potentially pathogenic, soluble IgM are regarded as transient, low affinity antibodies that first appear before the development of a memory B cell response that leads to production of mature, hypermutated high affinity IgG antibodies. Quite surprisingly, we found that some animals developed a vigorous IgM response that could be detected by ELISA, including those that had been infected with HHV6 B and some of the HHV6-A-infected monkeys (550-99 and 367-94). In sharp contrast, animal 190-94 which exhibited the most severe neuropathological lesions of inflammatory demyelination, failed to develop an IgM response as detected by ELISA (data not shown).

These results suggest that de novo exposure to HHV6 of certain individual marmosets (190-94, naïve to this virus as all animals were), results in the production of a particular IgG subclass (isotype) of antibodies that exclusively recognize conformational epitopes on one or more viral antigens (proteins, glycoprotein or lipid), and thus can only be detected by FACS and not by ELISA. These IgG antibodies develop in the absence of an ELISA-detectable IgM response against other epitopes. This pattern of antibody reactivity thus appears to be associated with development of inflammatory demyelination, as shown by neuropathological findings in animal 190-94 (FIGS. 7-8). On the other hand, all animals with milder or absent CNS disease, whether infected with HHV6 variant A or B, displayed a significant, and remarkably persistent IgM immune response against HHV6-Antigens that could be detected by ELISA.

While IgM reactivity detectable by FACS analysis and full characterization of the various antibodies with respect to epitope recognition and isotype switching is still under investigation, these findings indeed suggest that the nature and dynamic characteristics of the antibody response against HHV6 in individual subjects may profoundly influence, or even control the outcome and consequences of exposure to this virus in higher primate species. Specifically, it can be hypothesized that under genomic, post-genomic, post-transcriptional, and/or environmental influences or any combination thereof that result in failure to mount appropriate regulatory responses (for example, specific neutralizing antibodies), disease associated with viral persistence in CNS and/or other organs disease is permitted to develop in susceptible animals while repressed in the others. It is quite possible if not probable, that antibody isotype switching is a key regulatory mechanism of HHV6 infection and replication in vivo, in line of the recent observations that oligomerization of the CD46 viral receptor (Christiansen et al., J Virol 74:4672-4678 (2000)), or differential expression of the isoforms (CD46-cyt1 and CD46 cyt2) regulate the response of acquired immunity by controlling the phenotype of the CD4+ and CD8+ T cells (Evlashev et al., J Gen Virol 82:2125-2129 (2001); Marie et al., Nat Immunol 3:659-666 (2002); Kemper et al., Nature 421:388-92 (2003), regulatory cells, and NK cell activity (Grossman et al., Blood:Epub (2004)). It has recently been reported that peptide sequences from autoantibodies in lupus can influence the production of cytokines and the Th phenotype of an immune response. See, Kalsi et al., Lupus 13:490-500 (2004). Moreover and key to what is claimed in this application, it can be envisioned that in vitro studies of the host response to HHV6, or other virus may help predict the outcome of viral infection in vivo (e.g., clearance or persistence, and impact on the immune system). See, Ning et al., J Med Virol 69:306-312 (2003). Thus the cellular and/or antibody responses observed in vitro, or the characteristics of antibody responses against HHV6-Already present in vivo could be valuable biomarkers in terms of predicting a propensity and risk for individual subjects to develop MS or other autoimmune disorders prior to entering late childhood or adult life.

These observations are totally novel and of particular salience to clinical neurology and MS or chronic fatigue syndrome, because all studies to date have only utilized an ELISA method to detect either IgG or IgM antibodies against HHV6 in humans.

### Example 5

### Reactivity to CNS Myelin Antigens In Vivo

This Example discloses that in vivo infection with HHV6 induces immune system recognition of viral peptides homologous to an endogenous myelin peptide.

Viral infections with HHV6 resulted in molecular mimicry, a phenomenon by which the host's immune system recognizes a viral peptide that resembles a myelin protein peptide which triggers an immune attack. (Fujinami et al., Science 230:1043-1045 (1985); and Oldstone, Faseb Journal 12:1255-1265 (1998)). Such homology to an immuno-dominant peptide of MBP was recently described within the HHV6 U24 protein. Tejada-Simon et al., Ann Neurol 53:189-197 (2003).

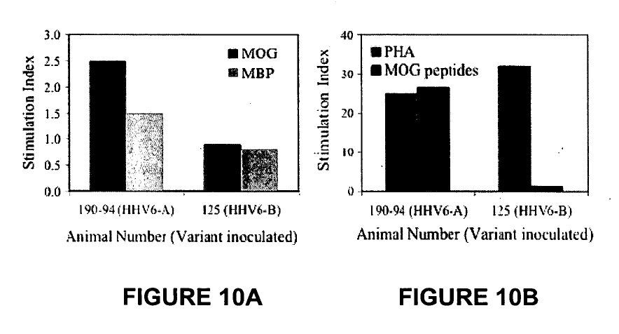

These data suggest that T cell mimicry occurs in HHV6-A-inoculated animals and that animals that displayed clinical signs and neuropathology had mounted significant T cell responses against MOG (extracellular domain, aa 1-125), MOG-derived peptides (a mixture of overlapping 20 amino acid peptides), and weak T cell responses to MBP. See, FIGS. 10A and 10B. No significant antibody (IgG) responses to myelin proteins were detected, in contrast to the typical humoral responses that occur in marmoset EAE. In contrast to myelin proteins, no significant T cell reactivity to viral lysates was detected in either PBMC or lymphoid organs.

Taken together, these data demonstrate that: (1) adult C. jacchus marmosets that were bred in captivity were naïve to HHV6-A and were infectable by this virus via hematogenous routes. Antibody responses against HHV6 develop after infestation; (2) repeated infection with HHV6-A produced a form of inflammatory CNS demyelination with neurological deficits and pathology similar to MS; and (3) MS is associated with de novo appearance of T cell reactivity to myelin antigens and persistence of virus in CNS lesions was necessary.

### Example 6

### Effects of Viral Infections on EAE

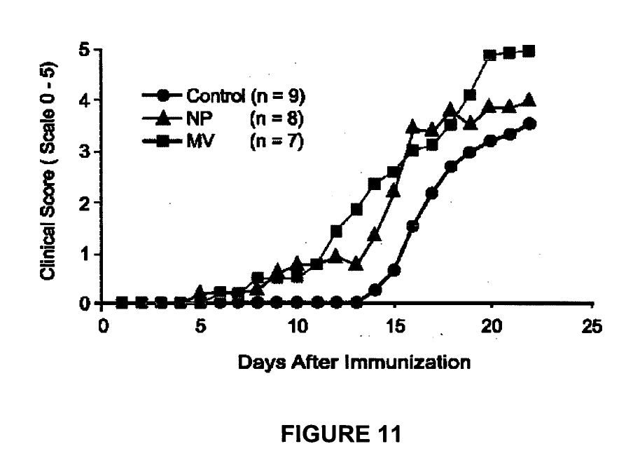

Similar to well-known effects of pathogen exposure in human autoimmunity, infection of laboratory animals prone to develop EAE worsens disease. Eralinna et al., J Neuroimmunol 55:81-90 (1994); Lieber et al., J Neuroimmunol 46:217-223 (1993); Massanari, Clin Immunol Immunopathol 19:457-462 (1981); and Mokhtarian et al., J Immunol 138:3264-3268 (1987). Mice expressing a TCR transgene that confers susceptibility to an encephalitogenic epitope of myelin fail to develop spontaneous or severe disease if raised in a pathogen free environment. Goverman et al., Cell. 72:551-560 (1993). Thus, in addition to HHV6's role in triggering mimicry and causing disease, these viruses have the potential to modulate the phenotypic expression of disease in MS. Similarly, additional experimental evidence indicated that exposure of adult wild type C57/Bl6 mice to either measles virus or its proteins aggravated symptoms of EAE induced with MOGaa35-55, an immunodominant epitope of MOG in rodents (Table 5 and FIG. 11).

TABLE 5

| Group | Incidence | | Maximum | |
|---|---|---|---|---|
| (# of animals) | of EAE | | Score ** | Day of Onset |
| Control (9) | 9/9 | 2/9 (22%) | 3.9 ± 1.6 | 20.0 ± 0.8 |
| Measles Virus (7) | 7/7 | 4/7 (57%) | 5.1 ± 1.2 | 15.7 ± 3.3 * |
| Measles NP (8) | 8/8 | 3/8 (38%) | 4.1 ± 1.7 | 18.9 ± 3.7 |

* p < 0.001 (Student's t test).
** mouse EAE scale 0 to 5. NP, recombinant measles virus nucleoprotein.

### Example 7

### Production of an MS-Like Illness in C. jacchus Marmosets by Exposure to HHV6

This Example discloses methodology for determining (1) whether a single or repeated exposure to HHV6 is necessary for disease expression; (2) which HHV6 variant(s) produce CNS autoimmune demyelination; and (3) what is the course of the associated disorder(s).

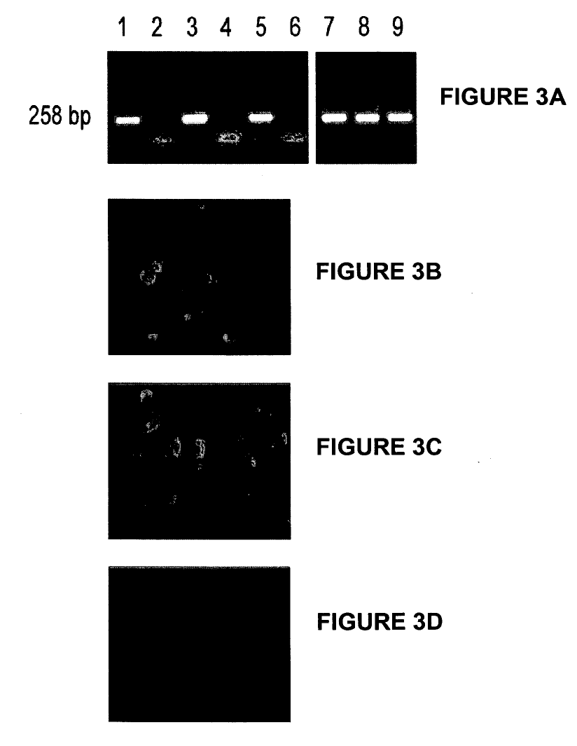

Two groups of 8 animals each are infected a first time with either HHV6-A or B, by intravenous injection of their own homologous infected PBMC. Freshly isolated PBMC are stimulated with 2.5 μg/ml phytohemagglutinin (PHA) and co-cultured in the presence of the respective infective cell lines in the transwell systems described in FIG. 3. Successful infection is assessed after 3-7 days by nested PCR using primers specific to amplify the major capsid protein gene DNA from each variant, and immunofluorescence (IFA) detecting the common nuclear antigen p41. Soldan et al., Nature Medicine 3:1394-1397 (1997) and Secchiero et al., J Clin Microbiol 33:2124-2130 (1995).

Five to 10×10⁶ infected PBMC are re-injected intravenously after thorough washing into their respective donors, and animals are monitored for 120 days for clinical and paraclinical markers of disease. If no apparent disease is observed at the end of this period, animals then receive a second injection of the appropriate viral lysate, and are monitored for up to 60 days. To investigate whether pure molecular mimicry rather than viral replication-induced cytotoxicity contributes to CNS pathology, a third group of six animals similarly receive two inoculations of homologous HHV6-A-infected PBMC after UV inactivation of the virus.

The latter experiments can be extended to three or more inoculations of inactivated virus, depending on when animals show evidence of immune reactivity to viral proteins. To control for non-specific factors, an additional six animals are injected with homologous, CMV or EBV-infected PBMC. Ablashi et al., Biomedicine 29:7-10 (1978). This methodology defines exposure requirements for production of CNS demyelinating disease, and identifies different disease phenotypes (e.g., acute, chronic-relapsing, and progressive) respectively associated with the two HHV6 variants. The methodology can be adjusted depending upon the results of initial observations of disease occurrence and course (e.g., multiple inoculations).

Animals are monitored daily for clinical signs of disease by observers blinded to the infection protocols. Blood and CSF are collected every 2 weeks for detection of CNS inflammation (CSF pleocytosis) (Genain et al., J. Clin. Invest. 94:1339-1345 (1994)), and in vitro investigations. Serial MRI imaging in C. jacchus can be employed in the event that no obvious clinical signs are evident.

One half of the animals are sacrificed at the acute stage of disease (i.e. within 7 days of onset), and the remaining animals in each group are observed for an additional 60 days in order to establish whether these protocols are capable of inducing chronic disease. All animals are sacrificed at the end of this period by exsanguination under deep pentobarbital anesthesia immediately followed by intracardiac perfusion with PBS then fixative while clamping the descending aorta, which preserves the thoracic and lumber portions of the spinal cord, and lower body lymph nodes and spleen which can be processed for cellular assays of immunological functions.

The entire neuraxis including optic nerves are collected and multiple specimens obtained and stored in fixed or frozen 2 mm sections. Samples are processed for routine histology, and future analysis by thin epoxy embedded sections, electron microscopy, and immunohistochemistry.

### Example 8

### Identification and Characterization of In Vivo Mechanisms for the Development of CNS Autoimmunity in the HHV6 Infected C. jacchus Marmoset Model System

The data presented herein, supra, demonstrated that (1) a mimicry-type of reactivity to myelin constituents developed in animals having clinical and/or neuropathological signs of disease; (2) viral replication at a distance from the acute infections was necessary in order for CNS destruction to develop; and (3) a two-stage infection may be necessary to produce this pathology.

T cell proliferative responses can be detected against myelin antigens (MBP, MOG, PLP, and 20-mer peptides) and viral lysates in serial PBMC samples, and in splenocytes and lymph node cells at euthanasia. Serum and CSF antibody reactivity (IgG and IgM) can also be tested by FACS analysis and IFA of HHV6-infected cell lines. If present, the nature of these responses (e.g., Th1 or Th2) can be analyzed by RT/PCR and ELISA of marmoset cytokines. See, Genain et al., Immunol. Reviews 183:159-172 (2001) and Genain et al., Science 274:2054-2057 (1996).

The identity of cell types responsible for T cell reactivity can be made using blocking antibodies (CD4+, CD8+). Proliferative responses are measured in the presence and absence of blocking antibodies to establish whether they are restricted by MHC class II and class I molecules.

Viral replication can be tested in serial samples of PBMC, serum and CSF, and in CNS and control tissues after euthanasia by PCR and immunohistochemistry (IHC), as described herein above. Soldan et al., Nature Medicine 3:1394-1397 (1997) and Secchiero et al., J Clin Microbiol 33:2124-2130 (1995). Assays of virus recovery are considered by co-culturing organ extracts with the uninfected HSB2 and MOLT 3 lines used to propagate HHV6 variants A and B in the laboratory.

Characterization of lesions is done by morphological studies and by immunohistochemistry (IHC). The cellular nature of inflammatory infiltrates (T cells [CD3+), CD4+, CD8+, B cells [CD20]; plasmocytes [CD38]); macrophages [HAM56, CD68], astrocytes [GFAP]); and cytokines are examined by RT/PCR and IHC (Th1: IL-2, TNF and IFN-γ; Th2: IL-4, IL-10, TGF-β). Oligodendrocyte and axonal pathologies are assessed using antibodies against MBP, PLP, MAG and MOG, anti-phosphorylated neurofilament antibodies and markers of apoptosis. Lucchinetti et al., Ann. Neurol. 47:707-717 (2000) and Trapp et al., J Neuroimmunol. 98:49-56 (1999). If evidence of oligodendrocyte or neuronal death is present, protocols will be developed to test the cytotoxicity of lymphocytes from infected animals towards primary cultures, and cell lines either transfected with myelin proteins or infected with HHV6.

Levels of expression of CD46 in CNS and other organs of infected animals and their respective controls are monitored. Levels of circulating soluble CD46 correlate with attacks of MS. Soldan et al., Ann Neurol 50:486-493 (2001). A panel of monoclonal and polyclonal antibodies that recognize marmoset CD46, recombinant human CD46, and CD46-transfected cell lines and controls are employed to explore whether this molecule can be used as a marker of disease activity.

HHV6-infected animals that develop CNS pathology do not mount a robust T cell response to the virus suggesting that these animals are unable to clear viral infection. immune dysregulation in MS may primarily involve a defect in a regulatory mechanisms that suppress autoimmunity in normal individuals (Antel et al., J Neuroimmunol 100:181-189 (1999)), and HHV6 clearance may be deficient in MS. Tejada-Simon et al., J Virol 76:6147-6154 (2002). In addition to T cell responses, this possibility is tested using a standard viral neutralization assay to detect HHV6 neutralizing antibodies in serum and CSF of infected animals.

The time-dependency of these analyzes is monitored in relation to appearance of clinical disease. For example, whether HHV6 infection is followed by acute monophasic, or chronic disease (relapsing or progressive) can be determined, and the correlation of these events with myelin- or HHV6-specific immune responses, and/or viral replication can be measured. Thus, intramolecular and intermolecular epitope spreading, a phenomenon that is observed in rodent EAE that has been proposed as a mechanism for relapses in MS can be detected. Miller et al., Immunol. Today 15:356-361 (1994) and Tuohy et al., Immunological Reviews 164:93-100 (1998).

### Example 9

### Exposure to Live Replicating HHV6 Viruses Influences the Course and Severity of EAE in Marmosets

Aggravation of MOGaa35-55-induced EAE infection of CD46 transgenic C57/Bl6 mice can be measured. Infection with sub-lethal doses of measles virus aggravates the severity of EAE in adult wild-type C57/Bl6 mice. Sensitization with viral proteins may be as effective as live measles virus, suggesting a possible mimicry mechanism. The susceptibility of adult cyt1 and cyt 2 CD46 transgenic mice to immunization with MOGaa35-55 in adjuvant can be determined. These animals can be infected with either measles virus or replicating HHV6 variants A and B via a peripheral injection prior to immunization. Control experiments can be performed using EBV or UV inactivated HHV6. Experiments can be conducted for periods of 30-45 days and can include attempts to re-infect animals by subsequent injections of virus.

Clinical and neuropathological endpoints can be used in mouse experiments. Analyses similar to the ones described for marmoset, which can be tailored depending on the occurrence of CNS disease and the various phenotypes observed. The mouse experiments can establish whether pathogenicity can be conferred to syngeneic recipients by adoptive transfer of cytotoxic lines and clones.

These models may be employed for expression profiling of CNS genes using microarrays. This technology is readily available in the art for mice and has been established with Agilent and Affymetrix human DNA microchips for marmoset tissues.

### Example 10

### Assay Systems for Measuring the Disease Phenotype Produced by HHV6 Infection in Mice Transgenic for Human CD46

Additional models of HHV6 infection in mice are employed to provide a comprehensive understanding of mechanisms of disease and for screening treatment strategies. Expression of CD46 is restricted in rodent species. Miwa et al., Immunogenetics 48:363-371 (1998). Mice expressing a human CD46 transgene were generated and shown to be infectable by measles virus. Kemper et al., Clin Exp Immunol 124:180-189 (2001); Evlashev et al., J Virol 74:1373-1382 (2000); and Oldstone et al., Cell 98:629-640 (1999). Several strains of mice that express two isoforms of human CD46 were generated that differ by the sequence of their cytoplasmic tail (cyt1 and cyt2) on a C57/Bl6 background that is susceptible to EAE induced with the MOG peptide aa35-55. Lyons et al., European Journal of Immunology 29:3432-3439 (1999). Signaling through these two isoforms differentially affects innate and acquired immunity in opposite fashions with regards to Th1 or Th2 preferences. Marie et al., Nat Immunol 3:659-666 (2002) and Ludford-Menting et al., J. Biol. Chem 277:4477-4484 (2002). CNS demyelinating pathology can be induced in these animals by productive infection with the HHV6 variants can be assayed.

Study groups of 10-15 animals each are performed in both cyt1 and cyt2 CD46 transgenic mice: (1) neonatal suckling mice (2-3 days old) are infected by direct intracranial injection (30 μl) of HHV6-A, HHV-B, and measles virus as a control. Titration experiments based upon the known lethal dose of measles virus are also performed.

Persistent infection in adult animals using infected PBMC is compared with intracranial injection. The effects of HHV6-A are separately analyzed, and the effects of single vs. two or more inoculations, as in the marmoset, are tested over a period of 45-60 days. Control experiments use EBV and UV inactivated HHV6 viruses.

### Example 11

### Immune Response to HHV6 in Multiple Sclerosis Patients and Unaffected Individuals

Immune dysregulation in multiple sclerosis primarily involves a defect in regulatory mechanisms that suppress autoimmunity in normal individuals. Antel et al., J Neuroimmunol. 100:181-189 (1999). The deficiency of HHV6 clearance in MS is assayed using a standard viral neutralization assay to detect HHV6 neutralizing antibodies in serum and CSF of patients that present with a clinically isolated syndrome (CIS), relapsing remitting MS (RRMS), and secondary progressive MS (SPMS). Tejada-Simon et al., J Virol 76:6147-6154 (2002). The inability of subjects susceptible to MS development, or other condition, following HHV6 exposure is further investigated by characterizing deficiencies in T suppressor (Ts) and T regulatory (Treg) cells, some of which are promoted via the CD46 cyt1 and cyt2 form signaling pathways.

### Example 12

### Glial Apoptosis and Chronic Relapsing Central Nervous System Autoimmune Demyelination Induced by HHV6

This example shows that C. jacchus marmosets, which are well known for their propensity to autoimmunity and susceptibility to experimental allergic encephalomyelitis (EAE), develop inflammatory demyelination following exposure to HHV6-A.

Common marmosets express a CD46 molecule highly homologous to the human receptor. Using trans-well cultures with HHV6-infected human T cell lines (HSB2 and MOLT3), it was found that marmoset peripheral blood mononuclear cells (PBMC) can be infected in vitro with both HHV6 variants A and B.

Nine (9) adult marmosets were infected with HHV6 in vivo, using various protocols, including: 1) intravenous inoculation of the animal's own PBMC infected in vitro with HHV6-A or HHV6-B (as verified by IFA and PCR), followed by intravenous injection of a cell lysate containing the same live virus 6-8 weeks later; 2) two intravenous injections of viral lysates from MOLT3 HHV6-B-infected cultures at 8 weeks interval; and 3) one inoculation of HSB2 cells infected with HHV6-A, followed by injection of either infected or uninfected cells 8-12 weeks later.

It was found that initial infection of the marmosets was nearly asymptomatic. Weight loss and hypotonic paralysis with sensory deficits in the marmosets that were repeatedly exposed to live HHV6-A virus (190-94, 550-99, U031-00, and U076-01) were observed. These marmosets were either male or females, and of adult age (1 to 9 years old).

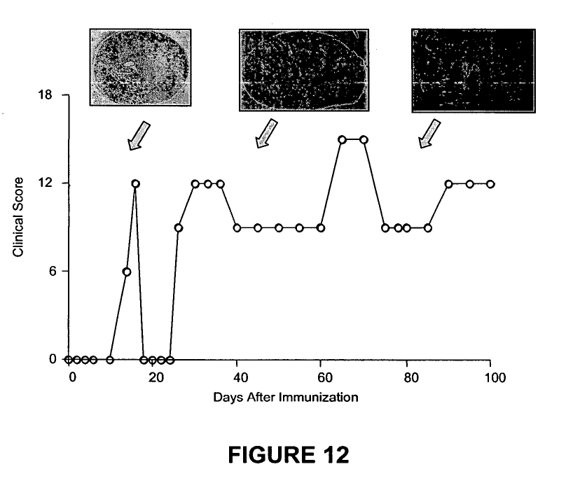

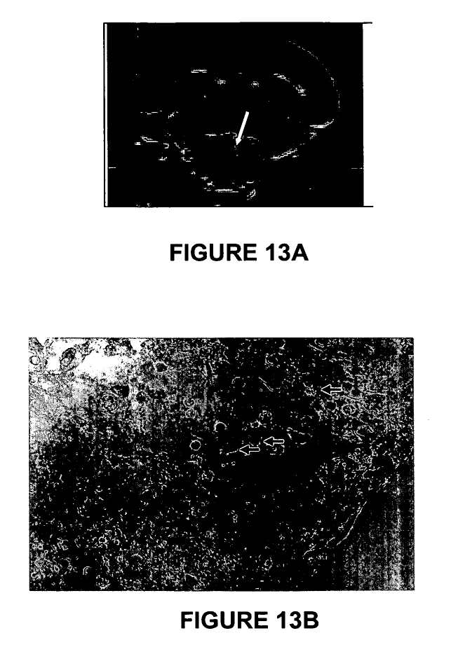

Hyper-intense T2-weighted lesions corresponding to perivascular infiltrates with inflammation and demyelination were observed in the marmosets that received live HHV6-A virus twice. FIG. 12 depicts an example of relapsing marmoset EAE with characteristic neuropathological features at each stage. These lesions were undistinguishable from those of marmoset EAE on routine histological stains as depicted in FIGS. 13A and 13B. FIG. 13A depicts hyper-intense T2 lesion in the marmosets' brain stem, adjacent to IV^{th} ventricle. FIG. 13B depicts demyelinating inflammatory infiltrate in the same animals (LFB/PAS).

The presence of HHV6 could be demonstrated by immunohistochemistry in inflammatory infiltrates as depicted in FIGS. 7A and 7B. Staining for early nuclear antigen p41/p38 demonstrate viral persistence/replication within lesions, in cells with the morphology of oligodendrocytes. Thus, appearance of CNS pathology may require viral persistence and/or replication. Numerous apoptotic cells were observed within lesions (TUNEL) of HHV6-A-infected animals, as depicted in FIG. 13B.

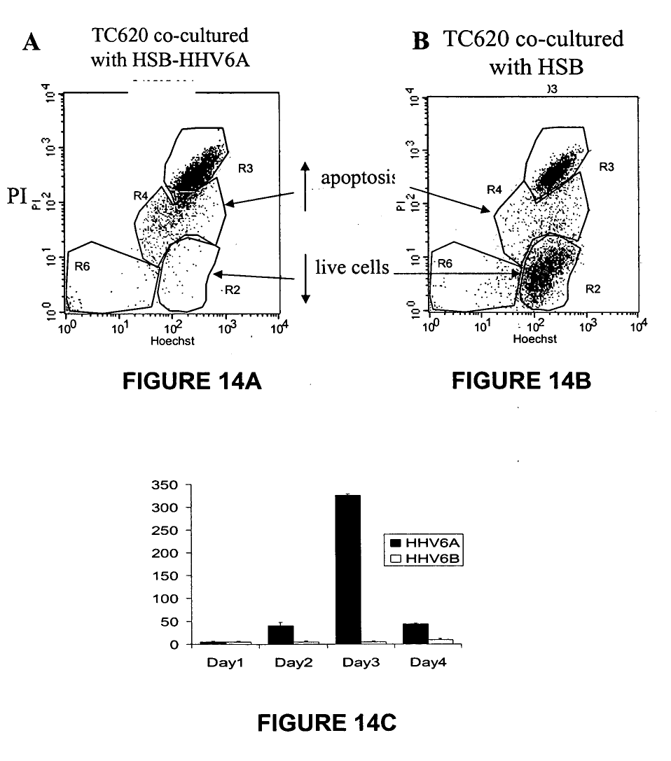

The human oligodendrocytoma cell line TC620 was used to test the hypothesis of a specific pro-apoptotic effect of HHV6 variants. FIGS. 14A and 14B depict the increase of apoptosis (R4) and decrease of live cells (R2) in TC620 cells co-incubated with HHV6-A-infected cell line (A) compared to the non-infected cell line (background, B). FIG. 14C depicts the percent increase of oligodendrocyte apoptosis observed after co-incubation with HHV6-A and HHV6-B infected cell lines. It was found that apoptosis is a specific effect of HHV6-A, not HHV6-B.

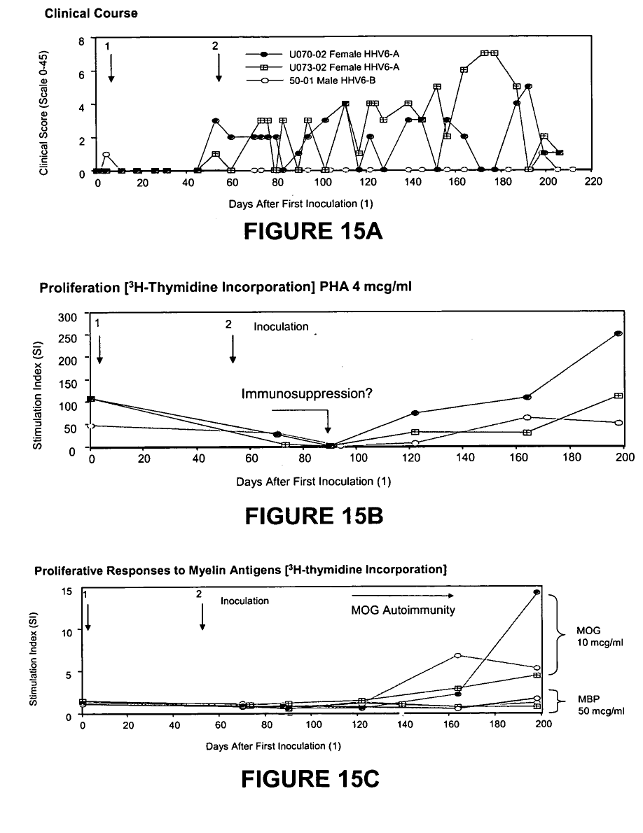

Two marmosets inoculated with HHV6-A, and one marmoset inoculated with HHV6-B, as control, were followed chronically to study the animals' relapsing course and reactivity to myelin antigens. The clinical course for these marmosets is depicted in FIG. 15A. Serial blood samples were obtained to measure peripheral T cell immune reactivity (PBMC) to phytohemagglutinin (PHA), myelin/oligodendrocyte glycoprotein (MOG), and myelin basic protein (MBP). The results are depicted in FIGS. 15B and 15C. The data shown in FIGS. 15B and 15C suggest that a second HHV6 inoculation is followed by a transient state of immunosuppression (decreased reactivity to PHA), and later by appearance of reactivity to MOG.

This experimental evidence show that C. jacchus marmosets are naïve to HHV6-A and B, and can reliably be infected by these viruses and that repeated infection with HHV6-A produces a mild, chronic relapsing CNS disease with pathologically, perivascular inflammatory demyelination similar to MS.

This model is the first to causally link a ubiquitous human virus to a chronic disorder mimicking MS; it affords model interactions between such microbes and complex neuro-immune responses in outbred species. It was found that HHV6 infection by both variants A and B may cause transient immunosuppression; both variants are capable of persistence and replication in marmosets, as in humans. However, only HHV6-A infestation results in MS-like CNS inflammatory demyelination suggesting a potential preferred CNS tropism for this variant and/or an apoptotic effect on glial cells. Further, it was found that mimicry with myelin antigens does not appear to be a primary or causal mechanism for inflammatory CNS damage in this model. Instead, delayed T cell auto-reactivity may play a role in perpetration of the chronic disease.

### Example 13

### Natalizumab-Induced Immunosuppression in a Case of Progressive Multifocal Leukoencephalopathy (PML)

Several cases of progressive multifocal leukoencephalopathy (PML) were recently reported in context of a clinical trial where patients with relapsing remitting multiple sclerosis (MS) received natalizumab in addition to interferon beta 1-a (IFN-β). Another patient treated with natalizumab for Crohn's disease, also developed a lethal form of PML. This example is directed to the immunological properties of lymphocyte subsets in a patient that developed progressive multifocal leukoencephalopathy (PML) after treatment with natalizumab and IFN-β.

Natalizumab is a humanized monoclonal antibody against the glycoprotein a4b1 integrin (very late antigen 4-VLA-4) expressed on the surface of T cells and monocytes. Experimentally, administration of natalizumab prevents cell adhesion to vascular endothelium and transmigration of lymphocytes across the blood brain barrier, a rationale for its therapeutic use in multiple sclerosis (MS). Anti-adhesion molecule approaches have proven efficient in murine models of inflammation, and in trials of human MS, with quasi-total abolition of MRI activity and clinical attacks.

PML is a severe, often rapidly lethal leukoencephalopathy that has been linked to a ubiquitous human virus (JC virus). The JC virus and related BK virus are thought to have evolved from the parent Simian Vacuolating virus (SV40), that contaminated the poliomyelitis vaccine administered to millions of Americans in the late 1950's. The first of these polyomavirus (papovaviridae family), the murine polyomavirus, was isolated by Gross in 1953 and shown to promote development of solid tumors. SV40 was isolated by Sweet and Hileman in 1960 in kidney cell cultures used to manufacture the Sabin oral polio vaccine. The JC and BK virus were isolated in 1971, respectively from a case of PML with Hodgkin's lymphoma and an immuno-supppressed kidney transplant patient. It was the use of human glial cells that afforded isolation of these 2 viruses, which underlines their preferred tropism. Polyomaviruses are small DNA viruses (around 5 kbp) and in addition to brain, have particular tropism for kidney cells and B cells. The receptor for SV40 appears to be MHC class I antigens. The JC virus does not appear to share this receptor with SV40, but may enter glial cells and other cell types via clathrin-dependent receptor-mediated endocytosis pits, and the serotonin receptor 5HT2AR. Like Herpesviridae, JC and BK viruses maintain a latent state of infection in man, but reactivate from time to time through life. Approximately 70-100% of adults have antibodies against JC virus and BK virus. The route of transmission is not known and there is no known animal reservoir. Most infections are asymptomatic, although some children may develop respiratory symptoms or cystitis.

PML is usually observed in immunosuppressed individuals (for example, AIDS, transplant patients), as are opportunistic infections with other common human pathogens. It is thought that B cells participate in the pathogenesis of PML by transporting the virus from kidney to brain, and that the disease is mediated through replication of JC virus in oligodendrocytes.

Heparinized blood and clotted serum were collected from: 1) one patient with ongoing PML, who had been treated with natalizumab monthly and IFN-β; 2) four additional patients treated with natalizumab and IFN-β in the same trial, who did not develop PML; 3) three patients with neuromyelitis optica (NMO), treated with steroids and plasma exchange; 4) five patients with relapsing remitting MS, treated with approved disease modifying therapies (interferon beta 1-b, interferon beta 1-a or copolymer 1) (MS-DMT); and 5) one healthy control individual.

All subjects were matched to the closest extent possible with the age of the patient that contracted PML. Whole blood was stained for flow cytometry (FACS) analysis according to the manufacturer's instructions. The following antibody clones were used: CD3-FITC/PE/PerCp: SP34, CD4-FITC/PE: L200 (BD Pharmingen), CD8-FITC: SFCI21Thy2D3 (Beckman Coulter), CD19-PE: 4G7, and CD25-FITC: 2A3 (Becton-Dickinson). ELISA was performed on serum for reactivity to MOG (extracellular domain). It was found that there was no difference in serum antibody reactivity between the different groups.

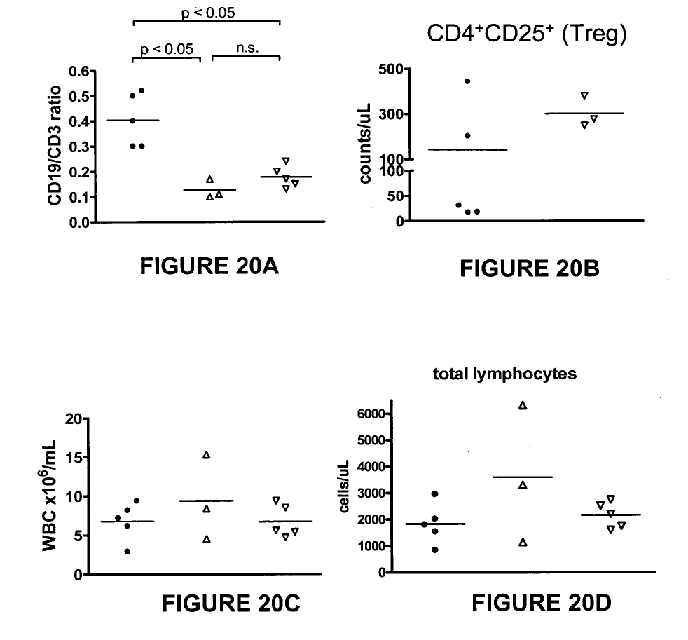

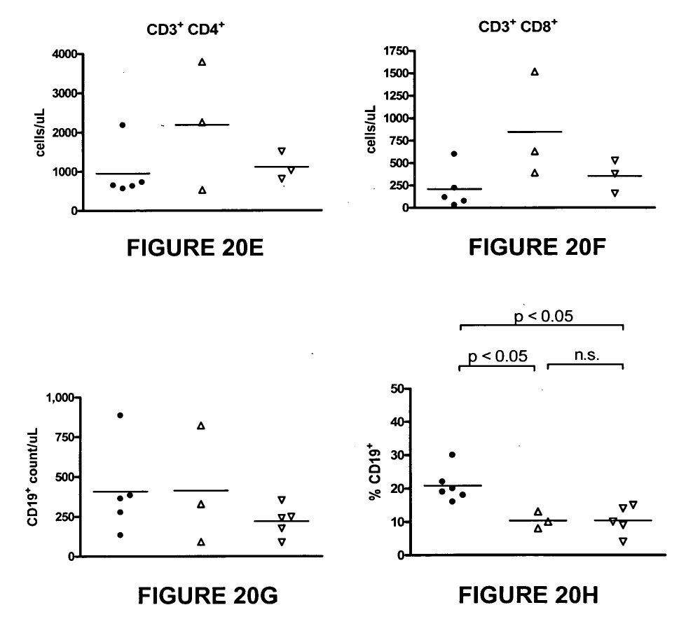

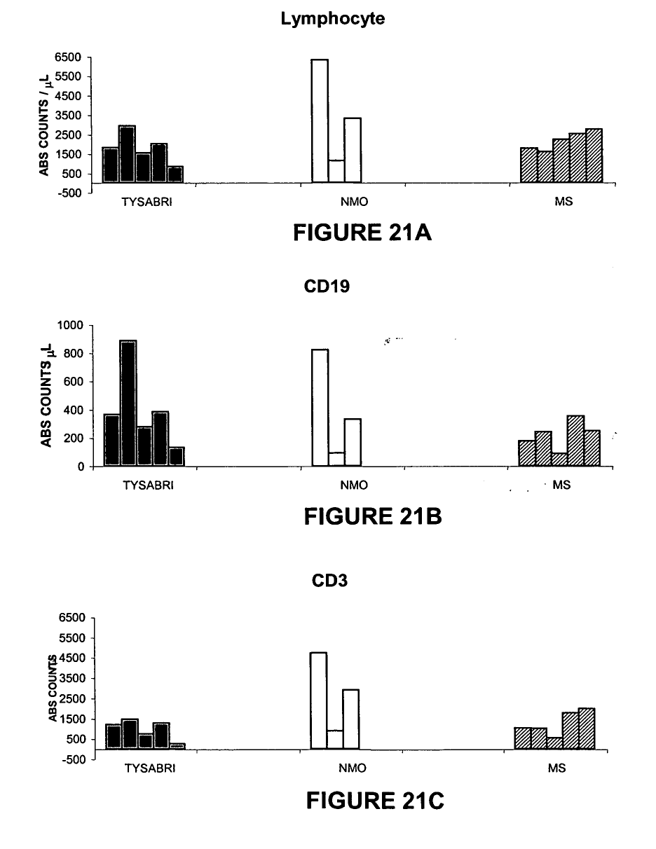

Table 6 shows the findings of flow cytometry studies. As shown in Table 4, there was no difference in total WBC, lymphocyte counts, and absolute counts of CD3⁺CD4⁺, CD3⁺CD8⁺, and CD 19⁺ cells. However, a trend was noted showing lower CD3⁺CD8⁺ cell counts in the patients treated with natalizumab+IFN-β compared to the other groups. FIGS. 20A through and 20H depict relative percentage of CD19+B cells and ratio of CD19⁺/CD3⁺ counts in patients treated with natalizumab+IFN-β (left), patients treated with NMO (center), and patients with MS treated with conventional DMT (right). Absolute CD19⁺ counts were also higher in those patients compared to the MS-DMT group, and the relative proportion of CD 19⁺ cells (mature B cells) was significantly increased (p<0.05). As a result, there was a significant difference between this group compared to the other NMO and MS patients when analyzing the ratio of absolute counts of CD19⁺/CD3⁺ cells, as shown in Table 4 and FIGS. 20A through 20H.

TABLE 6

| Group | Natalizumab + IFN-β | NMO | MS-DMT | P value* |
|---|---|---|---|---|
| WBC | 6800 ± 2500 | 9400 ± 5500 | 6700 ± 2100 | ns |
| Total Lymphocytes | 1830 ± 767 | 3587 ± 2602 | 2164 ± 490 | ns |
| CD3⁺CD4⁺ | 951 ± 691 | 2193 ± 1632 | 1114 ± 362 | ns |
| CD3⁺CD8⁺ | 209 ± 230 | 846 ± 593 | 354 ± 184 | 0.08 |
| CD19⁺ | 409 ± 127 | 414 ± 373 | 221 ± 98 | ns |
| CD19⁺(%) | 21.0 ± 4.9 | 10.3 ± 2.5 | 10.4 ± 4.4 | <0.05 |
| CD19/CD3 ratio | 0.40 ± 0.10 | 0.13 ± 0.04 | 0.18 ± 0.04 | <0.05 |
| CD4⁺CD25⁺ | 142 ± 185 | ND | 301 ± 68 | ns |

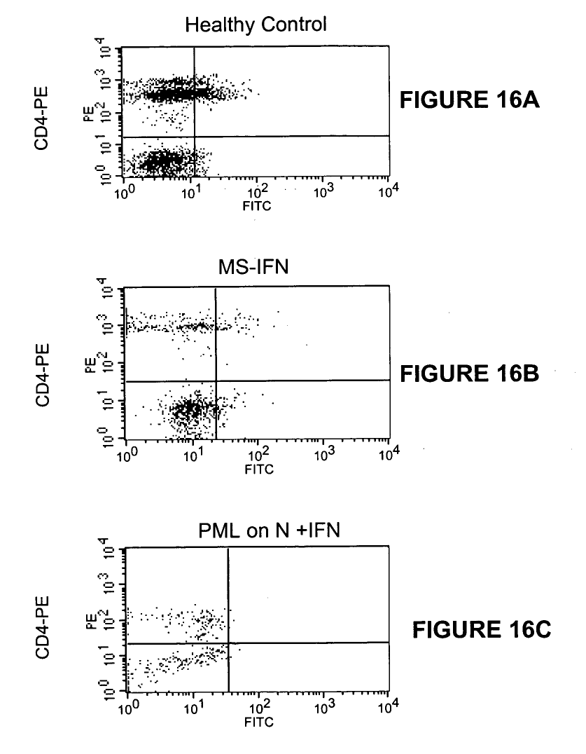

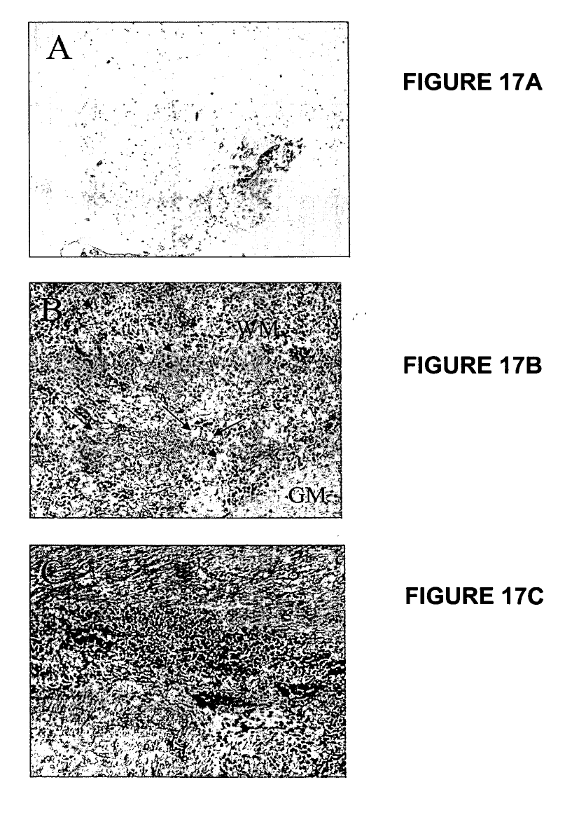

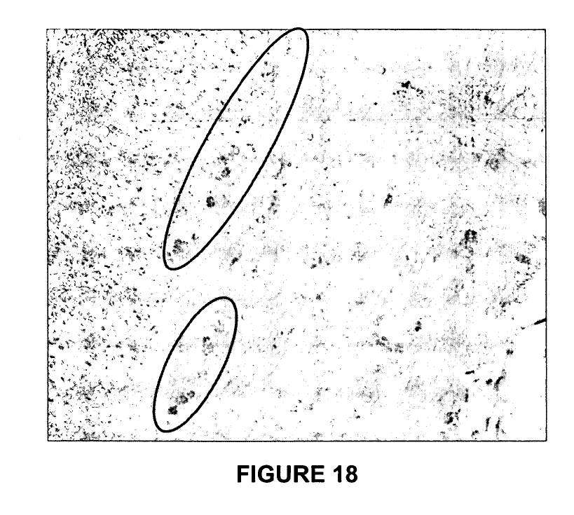

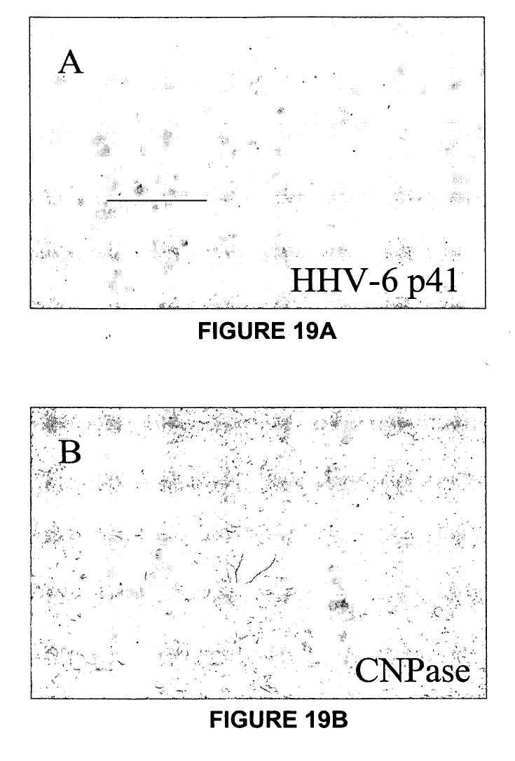

A small subset of T cells, the CD4+CD25+ cells, which are considered to include T cells with regulatory activity (Treg), were examined. These T cells express variable levels of CD25 in control samples, as shown in FIGS. 16A-C. FIGS. 16A-C depict representative flow cytometry data showing heterogeneous staining (low to high) for CD25 (FITC) in a healthy control (FIG. 16A), a patient with MS treated with IFN-β alone (FIG. 16B), and the patient receiving natalizumab+IFN-β that developed PML (FIG. 16C). Compared to the healthy control and patients on DMT, some patients treated with natalizumab+IFN-β exhibited a striking decrease of the Treg populations. Treg cells were virtually absent in these patients and in the subject that developed PML as a complication of this treatment. FIG. 20B depicts absolute counts of CD4+CD25+ cells in patients treated with natalizumab+IFN-β (left) and MS-DMT (right), as well as the subject that developed PML as a result of treatment with natalizumab+IFN-β.

This example demonstrates that treatment with natalizumab+IFN-β induces marked immune dysregulation in a subset of susceptible subjects. It was found that suppression of Treg populations occurs, despite relatively preserved total lymphocytes and CD4+ T cells and that B cell populations in these patients tend to increase, especially in proportion of CD8+ (suppressor) cells. In addition, it was found that loss of T regulatory activity may be responsible for deficient control of B cell activity and trafficking in these patients, and inability to prevent replication of dormant and usually benign viruses such as the JC virus. This state of “functional immunodeficiency” may also affect the ability of other ubiquitous pathogens to reactivate, although only cases of PML were observed. Further, it is possible that trafficking of infected B cells across the blood brain barrier may not be inhibited by natalizumab.

This study emphasizes the importance of understanding the consequences of strong and indiscriminate general immunosuppression in any treatment trial of MS. MRI may not be sensitive enough to detect early onset of complications such as PML, because it does not provide information on underlying pathology of T2-visible lesions. This data demonstrates that immunological markers such as T cell subsets, particularly CD19+ and CD4+CD25+, are useful for detecting patients at risk to develop natalizumab-induced complications. In particular, the data supports the assessment and use of CD19/CD3 ratios, CD4+CD25+ populations, CD8+ and CD3+ cell counts as well as counts of other immune cells including, for example, T regulatory cells, memory cells, NK cells, and their respective proportions as markers for monitoring the risks of patients with MS and other autoimmune disorders treated with anti-adhesion molecules such as natalizumab.

It will be appreciated that the approach described in the present invention will find wide application in methods for monitoring the risk of, for example, PML associated with a full range of viruses including, but not limited to, CMV, HSV, and other herpesviruses such as variants of HHV6, HHV7, and HHV8 as well as other opportunistic infections, in populations of patients with immunodeficiencies, constitutive or acquired, transplant patients, and AIDS and neuroAIDS.

What is claimed is:

1. A non-human animal model system for multiple sclerosis (MS) said non-human animal model system comprising a monkey and a herpesvirus wherein said monkey is infected with said herpesvirus.

2. The non-human animal model system of claim 1 wherein said monkey is selected from the group consisting of a marmoset, a New World monkey, and an Old World monkey, wherein said monkey is susceptible to infection with said herpesvirus.

3. The non-human animal model system of claim 2 wherein said marmoset is C. jacchus.

4. The non-human animal model system of claim 1 wherein said herpesvirus is selected from the group consisting of HHV6-A and HHV6-B.

5. The non-human animal model system of claim 1 wherein a single exposure of said monkey to said herpesvirus triggers and/or increases the severity of a central nervous system inflammatory disease.

6. The non-human animal model system of claim 1 wherein more than one exposure of said monkey to said herpesvirus triggers and/or increases the severity of a central nervous system inflammatory disease.

7. The non-human animal model system of claim 5 or claim 6 wherein said central nervous system inflammatory disease is multiple sclerosis.

8. The non-human animal model system of claim 1 wherein one or more exposure of said monkey to said herpesvirus, other herpes virus, or other virus triggers and/or increases the severity of other inflammatory diseases or malignancies of the central or peripheral nervous systems and neuromuscular junction selected from the group consisting of paraneoplastic syndromes and cerebellar degenration, limbic encephalitis, opsoclonus myoclonus, subacute sclerosing panencephalitis (SSPE), PML and other diffuse or focal leukodystrophies (early and late onset), acute and chronic polyneurpathies and polyradiculopathies, acute disseminated encephalomyelitis, myopathy, myasthenia gravis, Guillain Barre, miller-Fisher syndrome, Eaton Lambert syndrome, CNS vasculitis, sarcoidosis and neurosarcoid, Rasmussen's disease, paraneoplastic sensory neuropathy, CNS lymphoma, high and low grade oligodendroglioma and glioblastoma, glioblastoma multiformis, optic nerve glioma and meningioma, ependymoma and medulloblastoma.

9. The non-human animal model system of claim 1 wherein one or more exposure of said monkey to said herpesvirus or another virus results, triggers, and/or increases severity of other neurological disorders of unknown cause that include an inflammatory component selected from the group consisting of narcolepsy, chronic fatigue syndrome, stiff man syndrome, and autism in children.

10. The non-human animal model system of claim 1 wherein one or more exposure of said monkey to said herpesvirus triggers and/or increases the severity of an inflammatory disease and/or autoimmune disorder selected from the group consisting of diabetes, arthritis, anemia, lupus, pemphigus, thyroiditis, glomerular or intersticial nephritis, cardiomyopathy, myositis, dermatomyositis, hepatitis, and ulcerative colitis.

11. The non-human animal model system of claim 1 wherein said animal model system is suitable for the identification of factors mediating the direct toxicity of said herpesvirus towards a cell type selected from the group consisting of oligodendrocytes, astrocytes, and brain cells.

12. A transgenic mouse model system, comprising a transgene encoding CD46 and a herpesvirus, wherein said mouse is infected with said herpesvirus.

13. The transgenic mouse model system of claim 12 wherein said herpesvirus is selected from the group consisting of HHV6-A and HHV6-B.

14. The transgenic mouse model system of claim 12 wherein said transgene encoding CD46 is ubiquitously expressed in vivo.

15. The transgenic mouse model system of claim 12 wherein said transgene encoding CD46 is expressed in vivo in a tissue selected from the group consisting of brain, spinal cord, and peripheral nerve.

16. The transgenic mouse model system of claim 12 wherein a single exposure of said transgenic mouse to said herpesvirus triggers and/or increases the severity of a central nervous system inflammatory disease.

17. The transgenic mouse model system of claim 12 wherein more than one exposure of said transgenic mouse to said herpesvirus triggers and/or increases the severity of a central nervous system inflammatory disease.

18. The transgenic mouse model system of claim 16 or claim 17 wherein said central nervous system inflammatory disease is multiple sclerosis.

19. The transgenic mouse model system of claim 12 wherein one or more exposure of said mouse to said herpesvirus triggers and/or increases the severity of an inflammatory disease and/or autoimmune disorder selected from the group consisting of diabetes, arthritis, anemia, lupus, pemphigus, thyroiditis, glomerular or interstitial nephritis, cardiomyopathy, myositis, dermatomyositis, hepatitis, and ulcerative colitis.

20. The transgenic mouse model system of claim 12 wherein said model system is suitable for the identification of factors mediating the direct toxicity of said herpesvirus towards a cell type selected from the group consisting of oligodendrocytes, astrocytes, and brain cells.

21. The transgenic mouse model system of claim 20 wherein said factor is selected from the group consisting of CD4+ T-cells and CD8+ T-cells.

22. A non-human animal model system for the study of brain or spinal cord atrophy and degeneration in a disease affecting basal ganglia and gray matter said disease being selected from the group consisting of Alzheimer's disease, Parkinson's disease, Lewy body disease, Lafora disease, chorea and athetosis, Huntington's disease, and amyotrophic lateral sclerosis (Lou Gherig's disease).

23. A non-human animal model system for the study of the interaction between a virus and a primate immune system wherein said primate is selected from the group consisting of a human and a non-human.

24. A non-human animal model system for the study of the interactions between virus pairs wherein said virus pairs are selected from the group consisting of: (a) HHV6-A and HHV6-B; (b) HHV6-A and CMV; (c) HHV6-A and EBV; (d) HHV6-A and VZV; (e) HHV6-A and HHV8; (f) HHV6-A and HIV; (g) HHV6-A and HTLV; and (h) any one of HHV6-A, HHV6-B, CMV, EBV, VZV, and HHV8 and HIV.

25. An experimental system for the study of the potential of a candidate compound for reducing the severity of a disease, said experimental system comprising a herpesvirus infected non-human animal;
wherein said disease is selected from the group consisting of a demyelinating disease, a neurodegenerative disease, and multiple sclerosis; and
wherein said reduction in the severity of said disease is determined by measuring an inhibition of viral replication and/or transcription.

26. An experimental system comprising a mammal selected from the group consisting of a monkey, a wild-type mouse, an EAE mouse, and a CD46 transgenic mouse;
wherein said experimental system permits the testing of soluble CD46 (complement receptor) as a therapeutic agent.

27. A composition comprising a CD46 selected from the group consisting of (a) a soluble CD46, (b) a cell associated CD46, and (c) an artificial delivery system associated CD46;
wherein said composition is effective in reducing the severity of a disease selected from the group consisting of multiple sclerosis and/or other autoimmune and immune-mediated inflammatory diseases of the brain or other target organs;
wherein said soluble CD46 is produced in recombinant form, as a full-length polypeptide or as a truncated variants; and
wherein said artificial delivery system is either a liposome or a vesicle.

28. The composition of claim 27 wherein said composition is effective in the treatment of a neurodegenerative disorder and/or a tumor.

29. An experimental system for the study of a potential vaccine therapeutic for reducing the severity of a disease, said experimental system comprising a herpesvirus infected animal;
wherein said disease is an autoimmune and/or neurodegenerative disease.

30. The experimental system of claim 29 wherein said disease is multiple sclerosis.

31. The experimental system of claim 29 wherein said herpesvirus is HHV6.

32. A non-human animal model system for the early detection of an autoimmune and/or neurodegenerative disease prior to detectable disease onset in a patient.

33. The non-human animal model system of claim 32 wherein said patient is a child or teenager.

34. One or more methods for detection of certain antibodies against viruses such as, but not limited to HHV6 in serum, namely conformational and not limited to protein antigens, by means of fluorescence activated cell sorting analysis or other method where a detection tag is used to reveal presence of an antibody bound to its target antigen on the cell surface, or in other presentation where it resembles its native conformation.

35. Methods as above valued in their capacity to identify subjects where an active destructive process linked or concomitant to HHV6 replication and activity is ongoing, in order to initiate early treatment in these subjects and prevent full development of disease such as MS, chronic fatigue syndrome and other disorders.

36. A flow cytometric method for detecting in a pateint a viral infection comprising the step of detecting a virus-specific immunoglobulin responses wherein said virus is selected from the group consisting of HHV6, HHV7, HHV8, CMV, EBV, HSV, JC, BK, and SV40.

37. The methods of claims 34-36 where measurements of antibodies or in vitro cellular responses are used a biomarkers to predict individual risk for developing multiple sclerosis.

38. The method of claim 37 wherein the presence of said antibodies is predictive of a risk for developing a CNS disorder.

39. The method of claim 37 wherein the presence of said antibodies is predictive of a risk for developing an autoimmune disorder selected from the group consisting of diabetes, arthritis, anemia, lupus, pemphigus, thyroiditis, glomerular or interstitial nephritis, cardiomyopathy, myositis, dermatomyositis, hepatitis, and ulcerative colitis.

40. An experimental system for the identification of genes responsible for the development of an autoimmune and/or neurodegenerative disease following exposure to a herpesvirus, said experimental system employing a technique selected from the group consisting of a gene expression array, proteomics, metabonomics, and metabolonics.

41. An experimental system for the identification of genes responsible for the development of a detrimental autoantibody response that may lead to autoimmune and/or neurodegenerative disease following exposure to a herpesvirus, said experimental system employing a technique selected from the group consisting of a gene expression array, proteomics, metabonomics, and metabolonics.

42. An experimental system for the identification of genes responsible for the development of a beneficial autoantibody response (neutralizing antibody against virus) that may prevent development autoimmune and/or neurodegenerative disease following exposure to a herpesvirus, said experimental system employing a technique selected from the group consisting of a gene expression array, proteomics, metabonomics, and metabolonics.

43. A method for identifying a compound effective in reducing the severity of herpesvirus-mediated toxicity in the model system of claim 1 or claim 12 comprising the steps of (a) administering to said model system a candidate compound and (b) determining whether said herpesvirus-mediated toxicity is reduced in severity.

44. A method for evaluating the therapeutic value of compounds or other intervention that antagonize the development of detrimental autoantibodies as described in claim 1.

45. A method for evaluating the therapeutic value of compounds or other intervention that favor the development of beneficial autoantibodies as described in claim 1.

46. A methods and model system to evaluate the therapeutic value of compounds or intervention that alter the immune system via its cellular responses in the way to either antagonize detrimental autoantibodies or favor beneficial ones.

47. The method of claim 31 wherein said herpesvirus-mediated toxicity is correlative of a neurodegenerative disease selected from the group consisting of multiple sclerosis, Parkinson's disease, Alzheimer's disease, and cerebellar degeneration.

48. A method for detecting HHV-6 mediated cellular toxicity in a patient sample said method comprising the step of assaying cell death wherein said patient sample is selected from the group consisting of a CNS sample, a blood sample, and a CSF sample.

49. A flow cytometric method for assessing the risk of a patient developing a exhibit virus-related and cancerogenic complications following an immunotherapeutic treatment regimen, said method comprising the step of measuring an absolute CD3⁺CD8⁺ cell count, an absolute CD19⁺ counts, a relative proportion of CD19⁺ cells, and a CD19⁺/CD3⁺ ratio, wherein a reduction in CD3⁺CD8⁺ cell counts, an increase in absolute CD19⁺ counts, an increase in the relative proportion of CD19⁺ cells, and an increase in CD19⁺/CD3⁺ ratio indicates an increase the risk that a patient will exhibit virus-related and cancerogenic complications.

50. The flow cytometric method of claim 49 wherein said immunotherapeutic treatment regimen comprises a step of administering to said patient an antibody therapeutic selected from the group consisting of natilizumab, muromonab-CD3, abciximab, rituximab, daclizuniab, basiliximab, palivizumab, infliximab, trastuzumab, gemtuzumab, alemtuzumab, ibritumomab, adalimumab, omalizumab, tositumomab-I131, efalizumab, cetuximab, and bevacizumab.

51. A non-human animal model system for diabetes, said non-human animal model system comprising a monkey and a herpesvirus wherein said monkey is infected with said herpesvirus.

52. The non-human animal model system of claim 51 wherein said monkey exhibits a blood glucose level of between about 200 mg/dl and 2,000 mg/dl.
